(19) United States
(12) Patent Application Publication
(10) Pub. No.: US 2023/0219586 A1
(43) Pub. Date: Jul. 13, 2023

(54) TECHNIQUE FOR PROVIDING A USER-ADAPTED SERVICE TO A USER

(71) Applicant: 2HFUTURA SA, Panama City (PA)

(72) Inventor: Daniel GIERSCH, Monaco (MC)

(21) Appl. No.: 17/941,351

(22) Filed: Sep. 9, 2022

Related U.S. Application Data
(63) Continuation of application No. PCT/EP2021/057022, filed on Mar. 18, 2021.

(30) Foreign Application Priority Data
Mar. 18, 2020 (WO) PCT/EP2020/057449
Sep. 22, 2020 (WO) PCT/EP2020/076436

(51) Int. Cl. B60W 50/08; B60W 40/08

(52) U.S. Cl. CPC B60W 50/085; B60W 40/08; B60W 2050/0075

(57) ABSTRACT

A technique for providing a user-adapted service to a user is disclosed. A method implementation of the technique is performed by a computing system and comprises obtaining (S1002) a digital representation of personality data of a user, the personality data of the user being computed based on input regarding the user, wherein the input regarding the user includes actual personality information of the user, the actual personality information specifically relating to the user-adapted service and including at least one of: a current mood of the user at the time of providing the user-adapted service to the user, one or more preferences of the user specifically relating to the user-adapted service, and one or more goals of the user specifically relating to the user-adapted service; and processing (S1004) the digital representation of the personality data to provide a user-adapted service to the user.

### CROSS-REFERENCE TO RELATED APPLICATIONS

This application is a bypass continuation of prior Patent Cooperation Treaty (“PCT”) International Application No. PCT/EP2021/057022, filed Mar. 18, 2021, which claims priority benefit of each of PCT International Application No. PCT/EP2020/057449, filed Mar. 18, 2020 and PCT International Application No. PCT/EP2020/076436, filed Sep. 22, 2020. The entire disclosure of each of the applications cited in this section is hereby incorporated herein by reference.

### TECHNICAL FIELD

The present disclosure generally relates to the field of data retrieval. In particular, a technique for enabling efficient retrieval of a digital representation of personality data of a user by a client device from a server is presented. Moreover, a technique for providing a user-adapted service to a user of a client device is presented. The techniques may be embodied in methods, computer programs, apparatuses and systems.

### BACKGROUND

Personality tests have been used for decades to assess people's personality characteristics and are typically performed based on personality survey data obtained from a person to be tested, wherein the survey data is evaluated by a professional, such as a psychologist, to conclude on the person's personality. The so called “OCEAN” model is a widely accepted taxonomy for personality traits, also known as the “Big Five” personality traits, and includes openness, conscientiousness, extraversion, agreeableness and neuroticism as personality dimensions. Widely known personality tests using the OCEAN model include tests based on the so called International Personality Item Pool (IPIP), the HEXACO-60 inventory and the Big-Five-Inventory-10 (BFI-10), for example, which comprise sets of questions for testing a person on each of the five personality dimensions. As conventional personality tests generally require a review by a human professional, such as a psychologist, to obtain a qualified assessment of a person's personality traits, however, it is difficult to integrate carrying out personality tests and their results into processes performed on technical systems, although such integration could be beneficial because it would allow adapting processes to better fit a user's personality and, therefore, to improve user experience, such as by providing user-adapted services to the user, for example.

### SUMMARY

Accordingly, there is a need for a technical implementation which makes the integration of personality tests and their results into processes performed on technical systems practically feasible.

According to aspects of the present disclosure, a method, a computer program product and a client device for providing a user-adapted service to a user of the client device are provided according to the independent claims. Preferred embodiments are recited in the dependent claims.

According to a first exemplary aspect, a method for enabling efficient retrieval of a digital representation of personality data of a user by a client device from a server is provided, wherein the digital representation of the personality data is processed at the client device to provide a user-adapted service to the user. The method is performed by the server and comprises storing a neural network being trained to compute personality data of a user based on input obtained from the user, receiving, from the client device, a request for a digital representation of personality data for a user, and sending, to the client device, the requested digital representation of the personality data of the user, wherein the personality data of the user is computed using the neural network based on input obtained from the user.

By storing a trained neural network on the server and applying it for computing personality data of a user, retrieval of a digital representation of the personality data of the user may be made automatable (as conventional human reviews may no longer be needed) and, as such, an integration of the retrieval and use of users' personality data into (e.g., automated) processes performed on technical systems may become feasible. In particular, the neural network may be seen as an efficient functional data structure which enables computing the requested personality data in a single computational run, i.e., by inputting the input obtained from the user at the input nodes of the neural network and reading off the resulting output values representative of the personality data from the output nodes of the neural network. As such, the neural network may enable an efficient provision of personality data in the form of a digital representation to the client device, where it may be used to provide a service adapted to the particular personality of the user, to thereby improve user experience on the side of the client device. Due to the efficient provision of data, the integration of retrieval and use of personality data may especially become practical as the digital representation of the personality data may be provided to the client device without significant delay and may be processed at the client device instantly. A technical implementation may therefore be achieved which generally makes integrating the retrieval and use of personality data into processes performed on technical systems practically feasible.

The personality data of the user may be indicative of psychological characteristics and/or preferences of the user and, as such, the personality data may generally include psychological data as well as medical data (e.g., data indicating a tendency to curiosity, anxiety, depression, etc.), including classical personality data which may be based on the personality dimensions of openness, conscientiousness, extraversion, agreeableness and neuroticism (known as the Big Five, as described above), or personality dimensions of the conventional “16 personalities”, “Big Six”, or other established categorizations, for example. The digital representation of the personality data of the user may comprise a digital representation of the mentioned characteristics, such as a digital representation of at least one of the personality dimensions of openness, conscientiousness, extraversion, agreeableness and neuroticism, as computed by the neural network for the user, for example.

The client device may be configured to process the digital representation of the personality data for the purpose of enabling the provision of a user-adapted service to the user. In one variant, the client device may itself be configurable based on the digital representation of the personality data. An exemplary device which may be configurable by the digital representation of the personality data may be a vehicle, for example. The vehicle may in this case be the client device. The vehicle may process the received digital representation of the personality data of the user (e.g., a driver of the vehicle) and configure itself (e.g., including subcomponents thereof) so as to adapt the vehicle's driving configuration to the personality of the driver and to thereby provide a driving service that is specifically adapted to the personality of the user. If the personality data indicates that the driver tends to be risk-averse or anxious, for example, the vehicle's driving configuration may be configured to be more safety-oriented, whereas for drivers that tend to have a more risk-seeking personality, the vehicle's driving configuration may be configured to be more sporty. To this end, among other settings, the gas and brake reaction behavior of the vehicle may be adapted accordingly. Subcomponents of the vehicle providing vehicle-related services may be configured based on the personality data as well, such as a sound system of the vehicle including its sound and volume settings to better comply with the user's personality, for example. Optionally, the digital representation of the personality data may be shown to the user, giving the user the chance to modify at least one value of the digital representation of the personality data prior to providing the user-adapted service to the user, which may enable the user to vary the user-adapted service (at least to a certain extent) according to the user's current preferences.

In another variant, the client device may configure at least one other device based on the digital representation of the personality data, e.g., when it is the at least one other device that provides the service to the user. In such a variant, the client device may be a mobile terminal (e.g., a smartphone), for example, which may interface (e.g., using Bluetooth) with the vehicle (i.e., in this case, the vehicle corresponds to the at least one other device) and, upon receipt of the digital representation of the personality data from the server, the mobile terminal may configure the vehicle via the interface. It may thus be said that the digital representation of the personality data of the user may be processed at the client device to configure at least one device providing a service to the user. Configuring the at least one device may comprise configuring at least one setting of the at least one device and/or configuring at least one setting of a service provided by the at least one device. It will be understood that the vehicle is merely an example of a device which may be configurable based on personality data and that the client device and/or the at least one other device may correspond to other types of devices as well. Another example of a client device in such variant may be a server providing the user-adapted service to the user (at least partly) through a webservice or website, in which case the at least one other device may be a (computing) device which makes use of the webservice or the website to eventually provide the user-adapted service to the user.

In one implementation, the method performed by the server may further comprise receiving feedback characterizing the user, updating the neural network based on the feedback, and sending, to the client device, a digital representation of updated personality data of the user, wherein the updated personality data of the user may be computed using the updated neural network. The digital representation of the updated personality data of the user may be processed at the client device to refine a configuration of the at least one device providing the service to the user (e.g., one of the configurations of the vehicle mentioned above). The feedback may be gathered at the client device and/or at the at least one device providing the service to the user and may be indicative of the personality of the user. The feedback may include behavioral data reflecting behavior of the user monitored at the at least one device when using the service provided by the at least one device, for example, wherein, in one variant, the behavioral data may be monitored using (e.g., sensor based) measurements performed by the at least one device providing the service to the user. In the vehicle example, the behavior of the user being monitored may be the driving behavior of the user and the driving behavior may be measured by sensors at the vehicle, for example. For measuring the driving behavior, the sensors may sense the user's brake reaction and intensity, for example, and, since such measurements may be indicative of a user's personality (e.g., aggressiveness in driving), this information may be sent as feedback to the server in order to update the neural network and thereby refine the neural network's capability to compute personality data of users.

Updating the neural network may include training the neural network based on the feedback received from the client device, wherein, if the feedback represents a new input value which has not yet been input to the neural network, a new input node may be added to the neural network and the new input value may be assigned to the new input node when training the neural network. This makes the power of the neural network as an efficient functional data structure employed in the technical implementation presented herein especially apparent: the neural network represents an efficiently updatable data structure which may be updated based on arbitrary feedback on the user's personality received from the client device to refine its capability to compute personality data. The information conveyed by the feedback can be directly integrated into the neural network and may, once trained, immediately be reflected in subsequent requests sent to the server requesting digital representations of personality data. Conventional personality assessment techniques are rather fixed and may not support such updatability at all.

The digital representation of the personality of the user sent from the server to the client device may correspond to a digital representation of the personality of the user which was previously computed by the server upon a previous request for computing the personality of the user (e.g., upon performing a personality test by answering a set of questions by the user). The personality data of the user may thus be computed prior to receiving the request from the client device, wherein the request may include an access code previously provided by the server to the user upon computing the personality data of the user, wherein the access code allows the user to access the digital representation of the personality data of the user from different client devices. Such implementation may save computational resources at the server since the digital representation of the personality of the user may not have to be computed anew each time the digital representation of the personality data for that particular user is requested from a client device, but may be returned on the basis of the pre-computed personality data. The user, in turn, may use the access code to access the digital representation of personality data from a plurality of different client devices, such as from different vehicles the user may drive, e.g., a car and a motorcycle, or other types of devices.

The input obtained from the user may correspond to digital scores reflecting answers to questions regarding at least one of personality, goals and motivations of the user (as obtained in a question answer scheme in the manner of a personality test, for example; optionally, the questions may also comprise questions of an intelligence (“IQ”) test), wherein each digital score may be used as input to a separate input node of the neural network when computing the personality data of the user using the neural network. The digital scores may correspond to a five level Likert scale having values from 1 to 5, for example. The neural network may correspond to a deep neural network having at least two hidden layers between the input layer comprising the input nodes and the output layer comprising the output nodes of the neural network. The questions relating to personality may correspond to (or “comprise”) questions of the conventional IPIP, HEXACO-60 and/or BFI-10 pools, for example, but it will be understood that other questions regarding the personality of the user may be used as well, including questions on psychological characteristics, demographic characteristics and/or preferences of the user. Questions specifically relating to goals and motivations of the user may define additional dimensions (e.g., in addition to the Big Five) that may increase the accuracy of the computed personality data over the conventional IPIP, HEXACO-60 and BFI-10 techniques. The network may be trained based on data collected in a basic survey conducted with a plurality of test persons (e.g., 1000 or more), wherein the basic survey may be carried out using the questions mentioned above.

Exemplary questions beyond the conventional IPIP, HEXACO-60 and BFI-10 questions are shown in the tables presented herein below, wherein Table 1 provides an exemplary listing of questions specifically relating to motivations of the user, Table 2 provides an exemplary listing of questions specifically relating to goals of the user and Table 3 provides a listing of exemplary questions regarding other personality aspects of the user, including questions regarding demographic aspects of the user (e.g., questions 1 to 10 in Table 3), questions regarding preferences of the user (e.g., questions 11 to 15 in Table 3) and IQ test questions (e.g., questions 16 to 18 in Table 3). It will be understood that not all questions listed in the tables below may require answers that are directly mappable to a corresponding digital score, such as on a Likert scale, since the expected answers may be free text answers (e.g., questions 11 to 22 of Table 2 as well as questions of Table 3). It will be understood that one of skill in the art will be readily able to map such answers to corresponding digital scores as well, such as by correlating free text answers to predefined digital scores, for example. It will likewise be understood that, when it is mentioned above that the questions “correspond to” questions of the conventional IPIP, HEXACO-60 and/or BFI-10 pools, the questions may not need to literally use the exact wording of the predefined conventional questions, but may be rephrased as long as a semantic similarity or correspondence to the predefined conventional questions is maintained. The same likewise applies to the exemplary questions listed in the tables herein below.

The personality data of the user which is computed using the neural network may be taken as a “raw value” of the user's personality data. In some variants, the raw value of the user's personality data may be put in relation to personality data of a comparative group of persons (the comparative group containing a limited number of persons comprising at least one person, the personality data of the comparative group being calculated as averaged personality data among the persons in the group, for example) in order to obtain a “comparative value” (or “relative value”) of the user's personality data. In other words, the comparative value of the user's personality data may be obtained by measuring a distance (or difference) of the raw value of the user's personality data to the personality data of the comparative group (e.g., in each separate personality dimension). The distance (or difference) may then show the user's personality in comparison to the comparative group. As the comparative group may be selected differently depending on the use case (exemplary comparative groups may be “men only”, “women only”, certain “age groups”, “professional groups”, “education groups”, etc.), the comparative value of the user's personality data may vary depending on the use case accordingly. As a mere example, a user having a certain raw value in the extraversion dimension may have a high comparative value in the extraversion dimension in comparison to the user's family members, whereas the user may have a low comparative value in the extraversion dimension in comparison to the user's workmates.

In order to reduce the computational complexity when computing the personality data of the user, the neural network may be designed to have a specific network structure. In view of the context of the above questions, the structure of the neural network may generally be designed such that the number of input nodes is reduced as compared to the number of input nodes available when all of the above questions were used. The questions may thus correspond to questions selected from a set of questions representative of an optimally achievable result of computing personality data of a user (i.e., if all questions in the set of questions were answered by the user), wherein the selected questions may correspond to questions of the set of questions which are determined to be most influential with respect to the optimally achievable result. As each answer to a question may be input to a separate input node of the neural network, as described above, selecting a subset of the set of questions may reduce the number of input nodes when computing the personality data to thereby reduce the computational complexity. Due to the fact that questions which are most influential with respect to the achievable result are selected, the accuracy of the result output by the neural network may approximately be maintained.

In fact, tests have shown that the number of questions may be reduced drastically without significantly sacrificing result accuracy. Taking, as the set of questions being representative of an optimally achievable result of computing personality data, a set of questions which comprises the standard IPIP, HEXACO-60 and BFI-10 questions (summing up to a total number of 370 questions), optionally supplemented by further questions regarding goals and motivations of the user (resulting in a number of more than 370 questions in total), tests have shown that, when only the 30 most influential questions are used, approximately 90% of the accuracy of the optimally achievable result can be achieved. As such, the number of the selected questions may be less than 10% (preferably less than 5%) of the number of questions included in the set of questions being representative of the optimally achievable result. As, in this case, the number of input nodes of the neural network may be reduced extensively, computational resources may be saved significantly and personality data may be computed more efficiently.

In order to determine the questions of the set of questions which are most influential with respect to the optimally achievable result, in one variant, the questions may be selected from the set of questions based on correlating results achievable by each single question of the set of questions with the optimally achievable result and selecting questions from the set of questions which have the highest correlation with the optimally achievable result. A fixed subset of the set of questions being representative of the optimally achievable result may thus be determined, which may then be used to train the neural network with a reduced number of input nodes, as described above.

As said, the optimally achievable result may correspond to a result which is achieved if all questions in the set of questions were answered by the user, such as the set of questions comprising the standard IPIP, HEXACO-60 and BFI-10 questions, optionally supplemented by further questions regarding goals and motivations of the user, as described above. While, in one variant, the standard IPIP scores (as obtained by answering all questions in the standard IPIP test), the standard HEXACO-60 scores (as obtained by answering all questions in the standard HEXACO-60 test) and the standard BFI-10 scores (as obtained by answering all questions in the standard BFI-10 test) may be taken individually as reference for the optimally achievable result, in another variant, an improvement may be achieved by calculating a combined score of these individual scores as reference for the optimally achievable result, wherein the combined score may be calculated as a (e.g., weighted) average of the individual scores, for example. The combined score may also be denoted as a “superscore” representative of the “truth” derivable from the individual scores, generally improving the meaningfulness of the determined score and representing an improved reference for the optimally achievable result.

In another variant, the questions may be selected iteratively from the set of questions, wherein, in each iteration, a next question may be selected depending on an answer of the user to a previous question, and wherein, in each iteration, the next question may be selected as a question of the set of questions which is determined to be most influential on an achievable result for computing personality data of the user. This may be seen as an adaptive selection of the questions, wherein questions are determined user specifically in a stepwise manner taking into account answers to previous questions of the user. In one particular variant, the neural network may comprise a plurality of output nodes representative of a probability curve of a result of the personality data of the user, wherein determining the most influential question of the set of questions as the next question of the respective iteration may include determining, for each input node of the neural network, a degree according to which a change in the digital score which is input to the respective input node of the neural network changes the probability curve. The question associated with an input node for which the degree of change in the probability curve is determined to be highest may be selected as the most influential question for the respective iteration.

To further reduce computational complexity, the above iterative and adaptive selection may be performed under at least one constraint, such as at least one of a maximum number of questions to be selected, a minimum result accuracy to be achieved (the result accuracy may increase with each answered question per iteration and, when the desired minimal result accuracy is reached, the computation may be stopped), and a maximum available time (the test may be stopped upon lapse of the maximum available time, or each question may be associated with an estimated time to be answered by the user and the number of questions to be selected may be determined based on the estimated times). These constraints may be configurable for each computation of personality data separately.

According to a second exemplary aspect, a method for enabling efficient retrieval of a digital representation of personality data of a user by a client device from a server is provided. The method is performed by the client device and comprises sending, to the server, a request for a digital representation of personality data for a user, receiving, from the server, the requested digital representation of the personality data of the user, the personality data of the user being computed, based on input obtained from the user, using a neural network trained to compute personality data for a user based on input obtained from the user, and processing the digital representation of the personality data to provide a user-adapted service to the user.

The method according to the second aspect defines a method from the perspective of a client device which may be complementary to the method performed by the server according to the first aspect. The server and the client device of the second aspect may correspond to the server and the client device described above in relation to the first aspect. As such, those aspects described with regard to the method of the first aspect which are applicable to the method of the second aspect may be comprised by the method of the second aspect as well, and vice versa. Unnecessary repetitions are thus omitted in the following.

As in the method of the first aspect, the digital representation of the personality data of the user may be processed at the client device to configure at least one device providing a service to the user, wherein the at least one device may comprise the client device. The method performed by the client device may further comprise sending, to the server, feedback characterizing the user, and receiving, from the server, a digital representation of updated personality data of the user, wherein the updated personality data of the user may be computed using the neural network being updated based on the feedback. The digital representation of the updated personality data of the user may be processed at the client device to refine a configuration of the at least one device providing the service to the user. The feedback may include behavioral data reflecting behavior of the user monitored at the at least one device when using the service provided by the at least one device, wherein the behavioral data may be monitored using measurements performed by the at least one device providing the service to the user. The at least one device may comprise a vehicle, wherein the behavioral data may comprise data reflecting a driving behavior of the user. The personality data of the user may be computed prior to sending the request to the server, wherein the request may include an access code previously provided by the server to the user upon computing the personality data of the user, the access code allowing the user to access the digital representation of the personality data of the user from different client devices. The input obtained from the user may correspond to digital scores reflecting answers to questions regarding at least one of personality, goals and motivations of the user.

According to a third exemplary aspect, a computer program product is provided. The computer program product comprises program code portions for performing the method of at least one of the aspects mentioned above (including the first aspect and the second aspect) when the computer program product is executed on one or more computing devices (e.g., a processor or a distributed set of processors). The computer program product may be stored on a computer readable recording medium, such as a semiconductor memory, DVD, CD-ROM, and so on.

According to a fourth exemplary aspect, a server for enabling efficient retrieval of a digital representation of personality data of a user by a client device from the server is provided, wherein the digital representation of the personality data is processed at the client device to provide a user-adapted service to the user. The server comprises at least one processor and at least one memory, wherein the at least one memory contains instructions executable by the at least one processor such that the server is operable to perform any of the method steps presented herein with respect to the first aspect.

According to a fifth exemplary aspect, a client device for enabling efficient retrieval of a digital representation of personality data of a user from a server is provided. The client device comprises at least one processor and at least one memory, wherein the at least one memory contains instructions executable by the at least one processor such that the client device is operable to perform any of the method steps presented herein with respect to the second aspect.

According to a sixth exemplary aspect, there is provided a system comprising a server according to the fourth aspect and at least one client device according to the fifth aspect.

### BRIEF DESCRIPTION OF THE DRAWINGS

Further details and advantages of the technique presented herein will be described with reference to exemplary implementations illustrated in the figures, in which:

FIGS. 1a and 1b illustrate exemplary compositions of a server and a client device according to the present disclosure;

FIG. 2 illustrates a method which may be performed by the server according to the present disclosure;

FIG. 3 illustrates a method which may be performed by the client device according to the present disclosure;

FIG. 4 illustrates an exemplary interaction between a user, the server and a client device (exemplified by a car) according to the present disclosure;

FIG. 5 illustrates different connectivity options between a mobile terminal of the user, the car and the server according to the present disclosure;

FIGS. 6a and 6b illustrate exemplary structures of the neural network according to the present disclosure;

FIG. 7 illustrates an exemplary implementation which involves considering the driver's attention level to adapt settings of a vehicle according to the present disclosure;

FIG. 8 illustrates an exemplary implementation which involves considering body scan data of a user to provide a user-adapted service to the user according to the present disclosure;

FIG. 9 illustrates an alternative method which may be performed by the client device according to the present disclosure; and

FIG. 10 illustrates an alternative method which may be performed by a computing system according to the present disclosure.

### DETAILED DESCRIPTION

In the following description, for purposes of explanation and not limitation, specific details are set forth in order to provide a thorough understanding of the present disclosure. It will be apparent to one skilled in the art that the present disclosure may be practiced in other implementations that depart from these specific details.

Those skilled in the art will further appreciate that the steps, services and functions explained herein below may be implemented using individual hardware circuitry, using software functioning in conjunction with a programmed micro-processor or general purpose computer, using one or more Application Specific Integrated Circuits (ASICs) and/or using one or more Digital Signal Processors (DSPs). It will also be appreciated that when the present disclosure is described in terms of a method, it may also be embodied in one or more processors and one or more memories coupled to the one or more processors, wherein the one or more memories are encoded with one or more programs that perform the steps, services and functions disclosed herein when executed by the one or more processors.

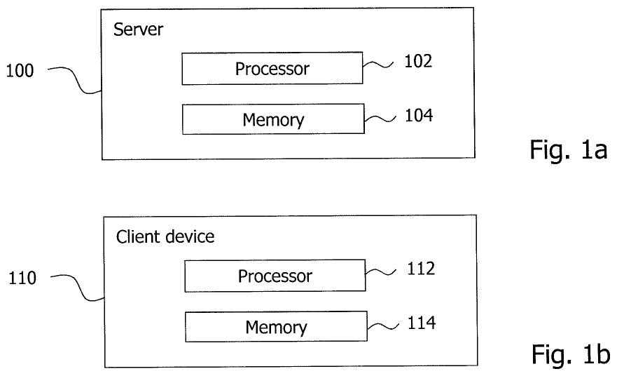

FIG. 1a schematically illustrates an exemplary composition of a server 100 for enabling efficient retrieval of a digital representation of personality data of a user by a client device from the server 100, wherein the digital representation of the personality data is to be processed at the client device to provide a user-adapted service to the user. The server 100 comprises at least one processor 102 and at least one memory 104, wherein the at least one memory 104 contains instructions executable by the at least one processor 102 such that the request server 100 is operable to carry out the method steps described herein with reference to the “server”.

It will be understood that the server 100 may be implemented on a physical computing unit or a virtualized computing unit, such as a virtual machine, for example. It will further be appreciated that the server 100 may not necessarily be implemented on a standalone computing unit, but may be implemented as components—realized in software and/or hardware—residing on multiple distributed computing units as well, such as in a cloud computing environment, for example.

FIG. 1b schematically illustrates an exemplary composition of a client device 110 for enabling efficient retrieval of a digital representation of personality data of a user by the client device 110 from a server. The client device 110 comprises at least one processor 112 and at least one memory 114, wherein the at least one memory 114 contains instructions executable by the at least one processor 112 such that the request client device 110 is operable to carry out the method steps described herein with reference to the “client device”. The client device may simply be denoted as a “client”. In some variants, the client 110 and the server 100 may be implemented on the same computing device (or computing system), wherein the client 110 and the server 100 may be implemented as components being executed on the same computing device/system, for example.

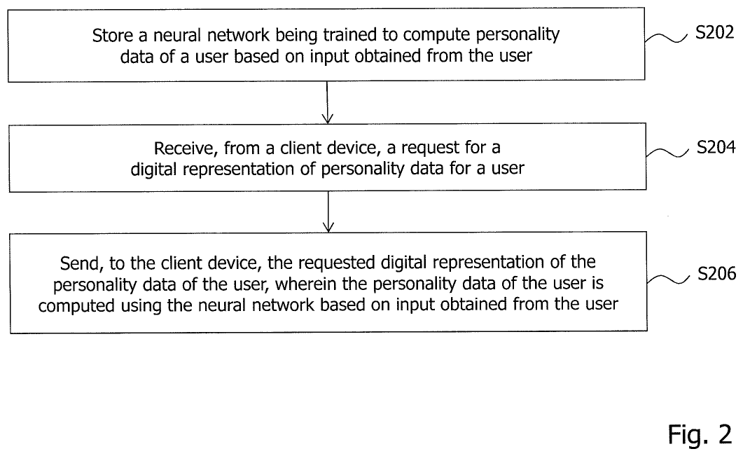

FIG. 2 illustrates a method which may be performed by the server 100 according to the present disclosure. The method is dedicated to enabling efficient retrieval of a digital representation of personality data of a user by a client device (e.g., the client device 110) from the server 100. In the method, the server 100 may perform the steps described herein with reference to the “server” and, in line with the above description, in step S202, the server 100 may store a neural network being trained to compute personality data of a user based on input obtained from the user, in step S204, the server 100 may receive, from the client device, a request for a digital representation of personality data for a user and, in step S206, the server 100 may send, to the client device, the requested digital representation of the personality data of the user, wherein the personality data of the user is computed using the neural network based on input obtained from the user.

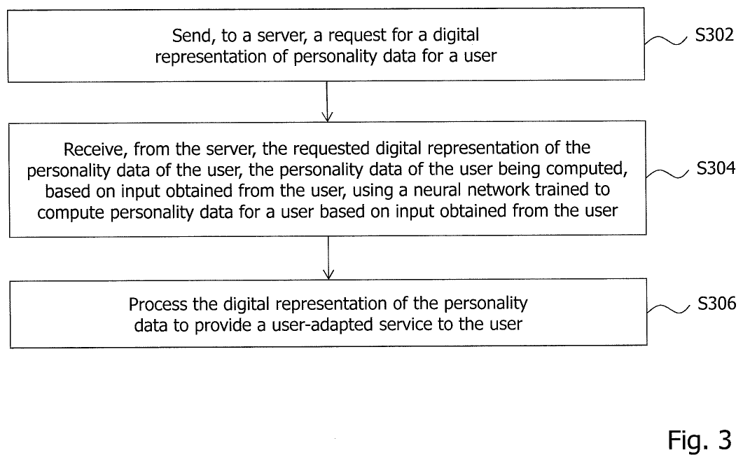

FIG. 3 illustrates a method which may be performed by the client device 110 according to the present disclosure. The method is dedicated to enabling efficient retrieval of a digital representation of personality data of a user by the client device 110 from a server (e.g., the server 100). In the method, the client device 110 may perform the steps described herein with reference to the “client device” and, in line with the above description, in step S302, the client device 110 may send, to the server, a request for a digital representation of personality data for a user, in step S304, the client device 110 may receive, from the server, the requested digital representation of the personality data of the user, the personality data of the user being computed, based on input obtained from the user, using a neural network trained to compute personality data for a user based on input obtained from the user and, in step S306, the client device 110 may process the digital representation of the personality data to provide a user adapted service to the user.

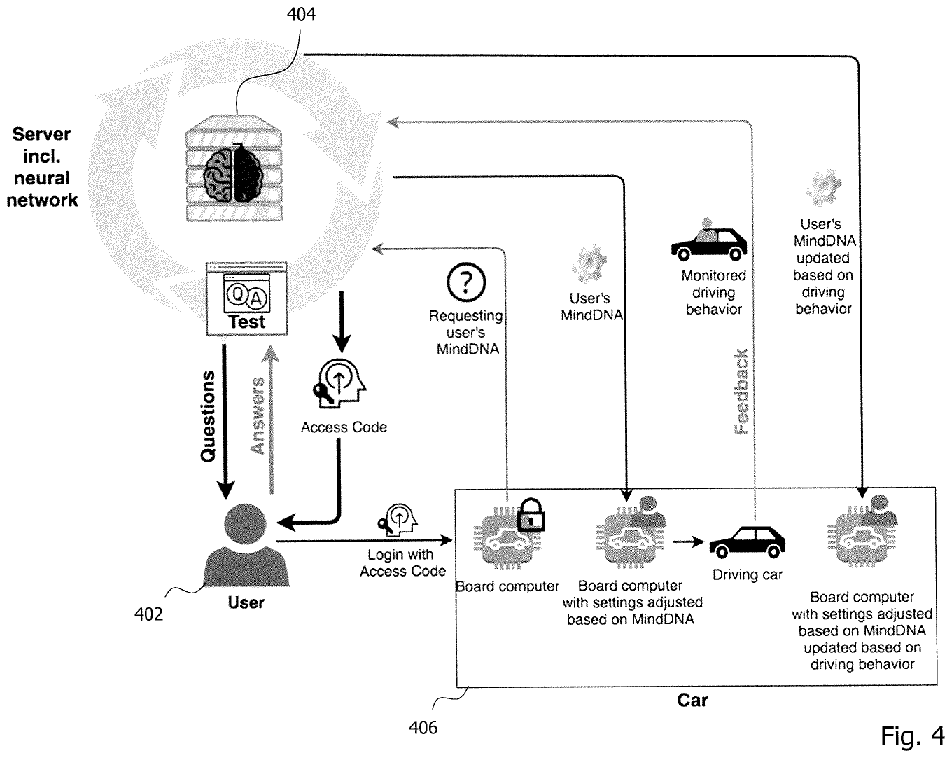

FIG. 4 illustrates an exemplary interaction between a user 402, a server 404 storing a neural network being trained to compute personality data of users based on input obtained from the users, and a client device for retrieving a digital representation of personality data of the user 402 to provide a user-adapted service to the user 402, wherein, in the shown example, the client device is a car 406 which may be driven by the user 402. As shown in the figure, the user 402 may perform an automated personality test by answering questions, e.g., using a web interface or an app on his laptop or smartphone, to thereby provide input to the neural network stored at the server 404 based on which the neural network may compute personality data for the user 402. Instead of sending a digital representation of the personality data to the user 402, in the shown example, the server 404 provides an access code to the user 402 which can be used by the user 402 to access the personality data using different client devices, including the car 406. The user 402 may register or login at the car 406 (more specifically, at its board computer) with the access code and the car 406 may then request, using the access code, the digital representation of the user's personality data from the server 404 (in the figure, the personality data of the user is denoted as the user's “MindDNA”).

Upon receiving the request from the car 406, the server 404 may return the user's personality data to the car 406, which may then configure its driving configuration (and, optionally, subcomponents of the car 406) in accordance with the personality data of the user 402, e.g., adapting the gas and brake reaction behavior of the car 406, to thereby provide a driving experience that is specifically adapted to the user's personality (e.g., risk-averse, risk-seeking, etc.). When the user 402 then drives the car 406, the car 406 may monitor the user's driving behavior, e.g., using sensors measuring the user's break reaction and intensity, and the car 406 may provide this information as feedback to the server 404, where the feedback may be processed to update (by training) the neural network to refine its capability of computing the personality data of the user 402. In response, the server 404 may send correspondingly updated personality data of the user 402 to the car 406 which may then use the digital representation of the updated personality data to refine the car configuration for a better alignment with the actual personality of the user 402. In sum, a system is therefore provided which may allow integrating retrieval and use of the user's personality data into an automated process to adapt the configuration of devices or services provided thereon in accordance with the preferences of the user derived his personality data, to thereby improve user experience.

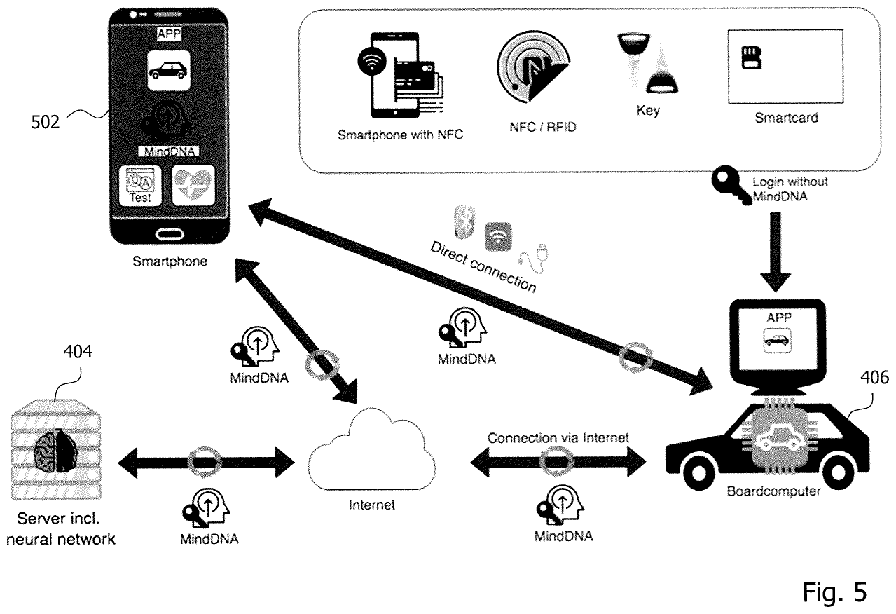

FIG. 5 illustrates different connectivity options between a mobile terminal 502 (e.g., a smartphone) of the user 402, the car 406 and the server 404 in accordance with the present disclosure. In one variant, the car 406 may communicate with the server 404 directly via the Internet and, upon authentication of the user 402 with the car 406 (e.g., using a key, smartcard, NFC/RFID, a smartphone with NFC, fingerprint, manually entered code, or the like), the car 406 may request the personality data of the user (in FIG. 5 again denoted as the user's “MindDNA”) to improve the driving experience of the user 402. In another variant, when the user 402 carries the mobile terminal 502, the mobile terminal 502 may (e.g., using a dedicated app installed thereon) communicate with the server 404 via the Internet and request the personality data of the user 402. In this variant, the car 406 may communicate locally with the mobile terminal 502 (e.g., using Bluetooth, Wi-Fi or USB cable) and retrieve the personality data of the user from the mobile terminal 502. A direct connection between the car 406 and the mobile terminal 502 may additionally be used to exploit sensors installed at the mobile terminal 502 (e.g., gyroscope for movement and acceleration detection, GPS for movement and acceleration detection as well as detection of driving routes, or medical sensors measuring pulse, blood pressure, or the like) to supplement the feedback gathered by the car 406 itself (e.g., in relation to the user's driving behavior) to thereby provide additional feedback sensed by the mobile terminal 502 to the server 404 for updating the neural network based on the feedback, as described above.

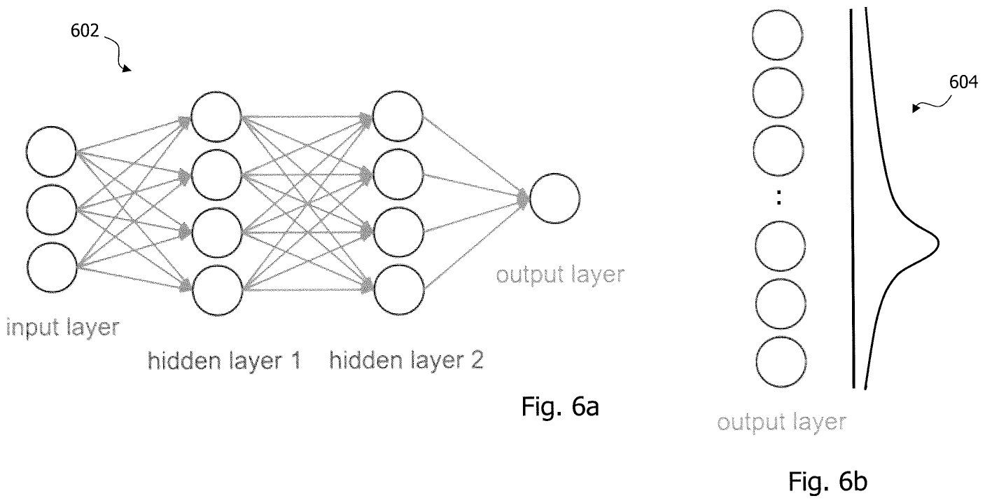

FIG. 6a illustrates an exemplary structure of a neural network 602 in accordance with the present disclosure. The neural network 602 comprises an input layer, an output layer and two hidden layers. It will be understood that the neural network 602 shown in FIG. 6a merely illustrates the structure of deep neural networks in general and that the actual number of nodes (at least in the input layer and the hidden layers) of the neural network 602 stored in the server 404 may be significantly higher than shown in the figure. As mentioned above, a test has been performed using the 30 most influential questions among a total number of 370 questions or more (taken from standard IPIP, HEXACO-60 and BFI-10 questions and, optionally, supplemented by further questions regarding goals and motivations of the user), resulting in 30 input nodes in the input layer of the neural network 602. In such a case, each of the hidden layers could be configured with 50 nodes, for example. Further, as shown in the figure, the neural network 602 may comprise a single output node in the output layer. In this case, the result value at the output node of the output layer may be representative of the value of one personality dimension (out of the Big Five) on which the neural network 602 has been trained. It will be understood that such structure of the neural network 602 is merely exemplary and that other structures are generally conceivable.

A more advanced structure of the neural network 602 comprises input nodes in accordance with the number of a full set of questions available, which may be taken from standard IPIP, HEXACO-60 and BFI-10 questions including further questions regarding goals and motivations of the user as well as still further questions on other psychological characteristics and/or preferences of the user not covered by the above questions, potentially adding up to several hundreds of questions, e.g., more than 600 questions. Such neural network 602 may thus have more than 600 input nodes, each corresponding to one of the questions of the full set of available questions, and the number of nodes of the hidden layers may be selected depending on the performance of the neural network 602. For example, the neural network 602 may comprise two hidden layers with 100 nodes each. Further, in the input layer, the above-mentioned more than 600 input nodes may be duplicated, wherein each duplicated input node may be used as a missing-question-indicator. The missing-question-indicators may be dichotomous, i.e., they may only have two values (e.g., 0 and 1) indicating whether the question of the corresponding (original) input node has been answered or not. Due to the duplicated input nodes, the input layer may comprise a total of more than 1200 input nodes.

The output layer of the more advanced neural network 602 may have a plurality of output nodes that together represent a probability curve for one personality dimension. If the scale used for the output in this personality dimension ranges from 0 to 10 and the number of output nodes is 50, for example, then each output node may be representative for a portion of the scale, i.e., corresponding to the portions 0-0.2, 0.2-0.4, 0.4-0.6, . . . 9.8.10 of the scale. Instead of a single output value, such output layer may deliver a whole probability curve for the output value on this personality dimension. FIG. 6b illustrates an exemplary output layer together with a corresponding probability curve 604. Such curve may allow determining where the output value most probably is (i.e., indicated by the peak of the curve) as well as determining the accuracy with which the neural network 602 calculates the result (i.e., indicated by the width of the curve). Using the advanced neural network 602, it may be possible to calculate the personality data of the user in the form of several probability curves (e.g., five probability curves corresponding to the Big Five) for an arbitrary number of answered questions, provided that the neural network 602 is trained separately for each dimension. In the initial state, in which no question has been answered yet, all missing-question-indicators may have the value of “missing” (e.g., 0). With every question which is then answered, an update of the output values may be calculated so that the width of the probability curves on the output layer becomes less with an increasing number of answered questions, so that the accuracy with which the neural network 602 calculates the result steadily increases.

Such structure of the neural network 602 may be particularly advantageous because it may allow iteratively selecting questions to be answered by the user next from the full set of questions, wherein, in each iteration, a next question may be selected depending on an answer of the user to the previous question, wherein, in each iteration, a next question may be selected as a question of the full set of questions which is determined to be most influential on an achievable result for computing personality data of the user. To this end, upon each answered question, the several (e.g., five) probability curves may be recalculated and, among the recalculated probability curves, the one which has the largest width (i.e., representing the probability curve currently having the at least accuracy) may be determined. As next question for the iteration, a question on this dimension may be selected to improve the accuracy on this dimension. In order to determine the most influential question, a degree according to which a change in the digital score input to the respective input node changes the probability curve (e.g., a degree in which the width of the curve changes) may be determined for each input node of the neural network 602. Based on this, the question associated with the input node for which the degree of change in the probability curve is determined to be highest may be selected as the most influential question for the respective iteration.

The advanced structure of the neural network 602 may also be advantageous because it may allow integrating feedback easily into the neural network. As described above, if the feedback represents a new input value which has not yet been input to the neural network 602, a new input node may simply be added to the neural network 602 and the new input value may be assigned to the new input node when training the neural network 602. In this way, any kind of new feedback may easily integrated into the network so that the neural network 602 may be refine its capability to compute personality data. As an implementation which reduces the computational complexity when adding a new input node, it may be conceivable that, when the network is trained to correlate the new input node with the other nodes of the network, only those nodes may be incorporated into the calculation which are determined to be most influential with respect to the optimally achievable result, to thereby avoid incorporating all nodes into the calculation. Also, it may be conceivable that, when the network is trained to correlate the new input node with the other nodes of the network, the number of layers being precalculated is limited (e.g., to 2 or 3) to avoid calculating all subsequent combinations of nodes, for example.

In the above description, the presented technique for efficient retrieval for a digital representation of personality data of a user has been exemplified in the context of adapting a vehicle's driving configuration, such as adapting the gas and brake reaction behavior of the vehicle to the personality of the user. In this case, the method described herein may also be denoted as a method for adapting a vehicle's driving configuration including an efficient retrieval of a digital representation of personality data of a user. It will be understood that adapting the gas and brake reaction behavior of the vehicle is just one example of adapting a vehicle's driving configuration and that, more generally, adapting the vehicle's driving configuration may comprise adapting any vehicle configuration that influences the driving behavior of the vehicle. Adapting the vehicle's driving configuration may as such comprise at least one of adapting a gas and brake reaction behavior of the vehicle, adapting chassis settings of the vehicle, adapting a driving mode of the vehicle, and adapting settings of an adaptive cruise control (ACC) of the vehicle, or the like, to the personality of the user. Adapting a driving mode of the vehicle may comprise setting an economy, comfort or sport mode to influence gas pedal and fuel consumption behavior of the vehicle depending on the driver's personality. If the personality data indicates that the driver tends to be risk-averse, for example, the driving mode may be set to economy or comfort, whereas for drivers that tend to have a risk-seeking personality, the driving mode may be set to sport mode. Adapting a drive mode of the vehicle may also comprise enabling/disabling an automatic four-wheel-drive (4WD) mode of the vehicle, for example. Adapting the settings of the ACC may comprise setting the distance to the vehicle ahead and/or the target driving speed, e.g., depending on the risk-averseness of the driver. For electric vehicles, adapting the vehicle's driving configuration may also comprise adapting the charging/discharging behavior of a vehicle battery (e.g., slow/fast charging, charging capacity level, slow/fast/uniform/non-uniform dissipation of energy) or adapting the simulated motor/exhaust sound produced by an external vehicle speaker (e.g., adapting sound type and/or equalizer settings of the corresponding sound system) depending on the personality of the user, for example. The charging/discharging behavior of the vehicle battery may likewise be reflected by adapting a charging/discharging behavior of a charging station accordingly.

It will be understood that the technique presented herein may also be employed for other purposes in a vehicle context, such as to adapt the environmental conditions in the passenger cabin of the vehicle (or, more generally, of a transport means, as an adaptation of the environmental conditions in the passenger cabin may similarly apply to other means of transport, such as aircrafts, trains, space shuttles, etc.). In this case, the method described herein may also be denoted as a method for adapting an environmental condition in a passenger cabin of a transport means including an efficient retrieval of a digital representation of personality data of a user. Adapting an environmental condition in a passenger cabin of a transport means may comprise adapting at least one of adapting a temperature of the passenger cabin (e.g., by adapting the air condition settings for the passenger cabin), adapting an internal lighting of the passenger cabin, and adapting an oxygen level in the passenger cabin (e.g., relevant for an astronaut in a space shuttle), or the like, to the personality of the user. Additionally or alternatively to adapting an environmental condition in the passenger cabin, the technique presented herein may also be employed to adapt user-specific settings regarding the passenger cabin. Adapting a user-specific setting regarding a passenger cabin of a transport means may comprise adapting at least one of adapting a seat configuration (e.g., seat height, seat position, seat massage settings, seat belt tensioning, etc.) for the user in the passenger cabin, and adapting equalizer settings of a sound system (e.g., increasing/decreasing basses or heights) provided to the user in the passenger cabin, or the like, to the personality of the user. With respect to a transport means having a plurality of seats for a multitude of passengers, such as a vehicle, train and aircraft, the technique presented herein may also be employed for seat allocation in the transport means. In this case, the method described herein may be denoted as a method for adapting a seat allocation in a passenger cabin of a transport means including an efficient retrieval of a digital representation of personality data of a user. Adapting a seat allocation in the passenger cabin may comprise allocating a seat to the user which is specifically adapted to the personality of the user (e.g., an open-minded and communicative user may be allocated to a seat next to other passengers e.g., at an aisle or middle seat, while an introverted user may rather be seated next to a window, for example). Upon allocating a seat to the user, a ticket may be issued and provided to the user (e.g., a printed train or aircraft ticket) allowing access to the allocated seat.

It will be understood that at least some of the above adaptations, i.e., adapting the vehicle's driving configuration, adapting the environmental conditions in the passenger cabin, and adapting the user-specific settings regarding the passenger cabin, may be performed adaptively in dependence from one another, i.e., if one setting is adapted manually or in consideration of the personality data of the user, this may automatically entail applying a set of further settings in consideration of the personality data of the user. For example, if the gas and brake reaction behavior of the vehicle is adapted to the personality of the user, this may automatically entail further adaptations, such as adapting the chassis settings and steering wheel settings accordingly. As another example, if the headlight of the vehicle is turned on for a cautious driver, 4WD and differential gears may automatically be activated as well. In yet another example, if the user turns on the heating system in the vehicle, steering wheel heating and/or seat heating may be turned on as well and configured to a heat level suitable for the user.

Any of the above adaptations of vehicle/transport means settings may—in addition to the adaptation to the personality of the user—also be performed in consideration of (or “based on”/“in accordance with”) sensor data indicative of a user's attention level obtained in the passenger cabin. In other words, the client device may be configured to adapt at least one of the vehicle's driving configuration, the environmental conditions in the passenger cabin, and the user-specific settings regarding the passenger cabin not only in consideration of the digital representation of the personality data of the user, but also in consideration of sensor data indicative of a user's attention level. The digital representation of the personality data of the user and the sensor data indicative of the user's attention level may in other words be combined prior to performing the above-mentioned adaptations. The sensor data indicative of the user's attention level may comprise data regarding at least one of the user's heartbeat, breath, tiredness, reaction time, and alcohol/drug level, for example. The sensor data may be collected by at least one sensor installed in the passenger cabin or in the mobile terminal of the user, for example.

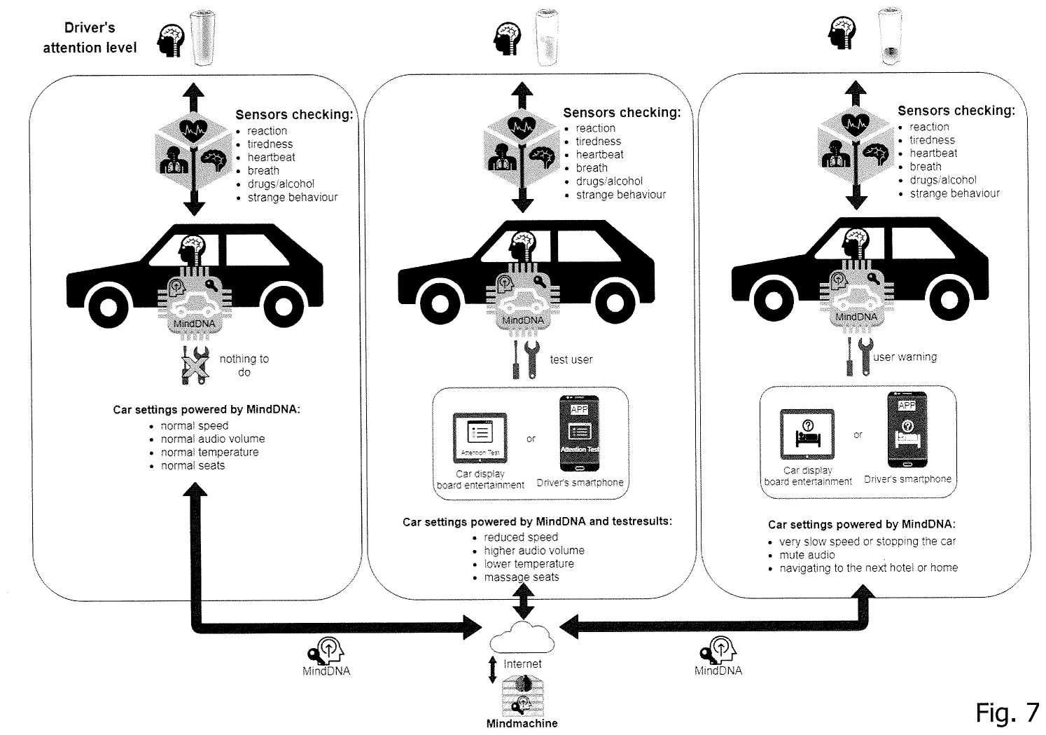

FIG. 7 illustrates an exemplary implementation which involves considering the driver's attention level in combination with the driver's personality data in order to adapt the vehicle's driving configuration, the environmental conditions in the passenger cabin and/or the user-specific settings regarding the passenger cabin. The driver's attention level may be checked by corresponding sensors in terms of the user's reaction time, tiredness, heartbeat, breath, alcohol/drug level, or unusual behavior of the user, for example. In the left portion of the figure, the collected sensor data is indicative of a normal attention level of the user and, hence, the vehicle settings may remain at the normal levels (e.g., as adapted to the driver's personality or “MindDNA”), including the speed, audio volume, temperature and seat settings, for example. In the middle portion of the figure, the sensor data is indicative of a reduced attention level of the driver and the vehicle settings may thus be changed to reduced speed, higher audio volume, lower temperature settings, including turning on seat massage features, in order to refresh the driver's attention again. Optionally, attention tests may be performed, such as requesting the driver to provide a voice-based response in a question/answer scheme, for example, and the results of the attention tests may be considered in adapting the above-mentioned settings. In the right portion of the figure, on the other hand, the sensor data is indicative of a very low driver's attention level and, therefore, a user warning may be provided and the vehicle settings may be adapted accordingly, e.g., to a very slow speed (and forcing to stop the vehicle at the next stopping opportunity, for example), to muted audio and/or to provide directions to the next hotel by a navigation system, for example.

The above adaptations of vehicle/transport means settings may also be performed in consideration of (or “based on”/“in accordance with”) at least one of geographical data, weather data and time data regarding a planned route to be traveled using the vehicle or transport means. In other words, the client device may be configured to adapt at least one of the vehicle's driving configuration, the environmental conditions in the passenger cabin, and the user-specific settings regarding the passenger cabin not only in consideration of the digital representation of the personality data of the user, but also in consideration of geographical data, weather data and/or time data regarding the planned route. The digital representation of the personality data of the user and the additional data regarding the planned route may in other words be combined prior to performing the adaptations. The geographical data may comprise data on the topography of the planned route, such as ascending/descending gradients of mountain roads, information on serpentine or coastal roads, altitude, or the like. The weather data may comprise information on current weather conditions (as sensed by the vehicle or transport means itself, e.g., using a rain sensor, temperature sensor, etc.) or information on forecast weather conditions for the planned route (e.g., rainy, cloudy, sunny, etc.). The time data may comprise information on a time schedule for the planned route, such as driving during the day, driving during light-transition periods (dusk or dawn) or driving during night, for example. Depending on such data, the vehicle's driving configuration, the environmental conditions in the passenger cabin, and the user-specific settings regarding the passenger cabin may be adapted to better fit the users personality, such as to activate 4WD in order to provide a safer driving experience for a risk-adverse driver in case of difficult topographic/weather/time conditions along the planned route, for example.

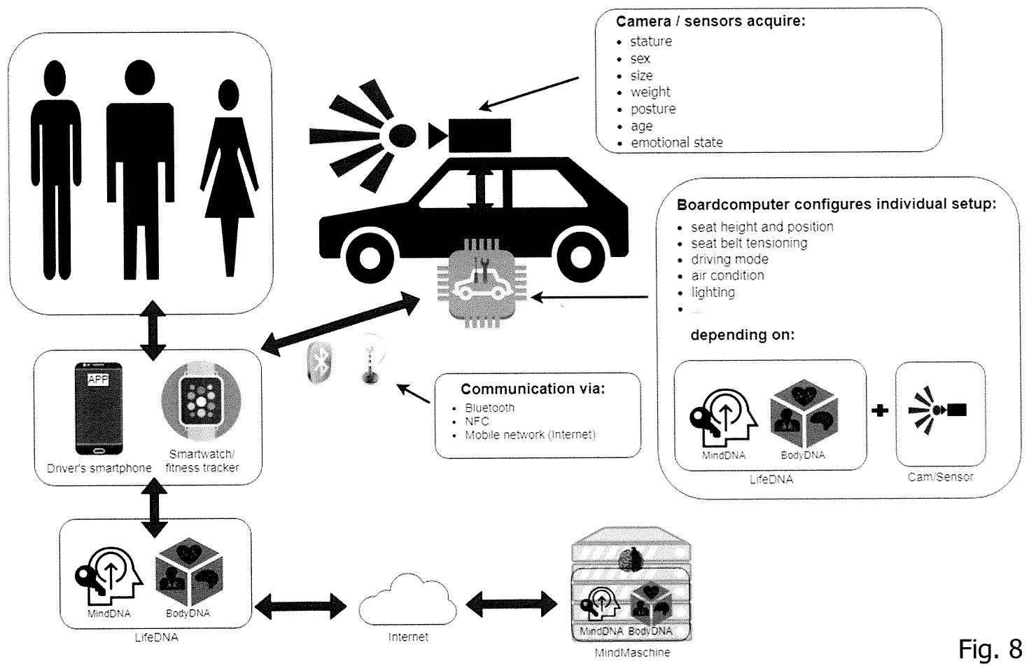

In order to provide a user-adapted service to the user, as described above (e.g., by adapting at least one of the vehicle's driving configuration, the environmental conditions in the passenger cabin, and the user-specific settings regarding the passenger cabin), the client device may further consider body scan data indicative of (e.g., physical) characteristics of the user derivable by scanning (e.g., at least a portion of) the user's body prior to providing the user-adapted service to the user (e.g., prior to the user driving the vehicle). The user characteristics which are derivable by scanning the user's body may include at least one of the user's size, weight, sex, age, stature, posture, and emotional state, for example. Alternatively or additionally, the user characteristics derivable by a body scan may also include certain movements of the user or items carried by the user, for example. The body scan data may be obtained by a radar device, camera or voice recorder (e.g., of the mobile terminal of the user, or installed at the vehicle/transport means; including 360 degree cameras, infrared (IR) cameras, etc.) acquiring one or more images or speech signals of the user, wherein body/face/voice recognition techniques may be employed to scan the user's body and derive the user characteristics mentioned above. The client device may thus be configured to provide a user-adapted service not only in consideration of the digital representation of the personality data of the user, but also in consideration of (or “based on”/“in accordance with”) the body scan data. The digital representation of the personality data of the user and the body scan data may in other words be combined prior to providing the user-adapted service to the user. FIG. 8 illustrates an exemplary implementation which involves considering a driver's body scan data (e.g., obtained by the driver's mobile terminal, such as the driver's smartphone, smartwatch or fitness tracker, prior to entering the vehicle) in combination with the personality data of the driver in order to adapt the vehicle's driving configuration, the environmental conditions in the passenger cabin and/or the user-specific settings regarding the passenger cabin accordingly. In the figure, the body scan data is denoted as “BodyDNA”, which in combination with the “MindDNA”, forms the so-called “LifeDNA”. It will be understood that the obtained body scan data may also be used to provide feedback characterizing the user to update the neural network, as described above.

It will further be understood that, in other implementations, it may also be conceivable that the client device is configured to provide the user-adapted service in consideration of the body scan data only, i.e., without consideration of the digital representation of the personality data of the user. In such an example, the body scan may detect the user (e.g., using face recognition for authentication purposes) and open the door of the vehicle when the movement of the user (as determined by the body scan) indicates that the user approaches the vehicle. Likewise, when it is detected that the user carries an item (e.g., a bag or suitcase), the trunk of the vehicle may be opened automatically, for example. Such method may generally be phrased as a method for providing a user-adapted service to a user, the method being performed by the client device and comprising obtaining body scan data indicative of characteristics of the user derived by scanning at least a portion of the user's body, and processing the body scan data to provide a user-adapted service to the user. Any of the exemplary body scan data mentioned above may be used for such purpose and, in case of the client device being a vehicle, the body scan data may be used (i.e., without further consideration of personality data of the user in the above sense) to adapt at least one of the vehicle's driving configuration, the environmental conditions in the passenger cabin and the user-specific settings regarding the passenger cabin, for example. It will be understood that, if at least part of the body scan data is already available (e.g., pre-stored) in a user profile of the user, such data may also be obtained from the user profile upon authenticating the user, in which case a body scan to determine corresponding data may not be necessary to be performed in real-time. What is described in this paragraph, i.e., that the client device may be configured to provide the user-adapted service in consideration of the body scan data only, i.e., without consideration of the digital representation of the personality data of the user, may likewise be applicable to other vehicle-related use cases described herein, including the use case which takes into consideration sensor data indicative of a user's attention level, the use case which takes into consideration at least one of geographical data, weather data and time data regarding a planned route to be traveled described above, as well as the use case which takes into consideration predefined conditions being monitored and being potentially indicative of a suicidal intent of the user, and the use case which takes into consideration goals and/or preferences of users driving in other vehicles the vicinity to implement a collectively enhanced driving behavior of a group of vehicles described below, for all of which it is generally conceivable that they likewise operate without additional (or “combined”) consideration of the digital representation of the personality data of the user.

In another vehicle-related use case, the technique presented herein may also be used to determine a vehicle configuration that is adapted to the personality of the user prior to manufacturing the vehicle, wherein the vehicle may then be manufactured at least partly based on (or “in accordance with”) the determined vehicle configuration. The vehicle may be manufacturable in different configuration options (e.g., as offered by a vehicle manufacturer), such as with different motor options each having a different motor power, drive technology options (e.g., support of two-wheel-drive (2WD) or 4WD technology), chassis options, different drive mode options, support of ACC, etc., and when a new vehicle is to be manufactured for the user, the vehicle configuration may be determined to be specifically adapted to the personality of the user. For example, if the personality data indicates that the user tends to be risk-averse, the determined vehicle configuration may comprise a selection of a motor having a lower power as compared to a vehicle configuration determined for a user whose personality data indicates a risk-seeking personality. Based on the determined vehicle configuration, the vehicle may then be manufactured accordingly. As such, in line with the above description, it may also be envisaged a method for vehicle manufacturing including an efficient retrieval of a digital representation of personality data of a user by a client device from a server, the digital representation of the personality data being processed at the client device to provide a vehicle configuration adapted to the personality of the user. The method may comprise sending, from the client device to the server, a request for a digital representation of personality data for a user, receiving, by the client device from the server, the requested digital representation of the personality data of the user, the personality data of the user being computed, based on input obtained from the user, using a neural network trained to compute personality data for a user based on input obtained from the user, processing the digital representation of the personality data to determine a vehicle configuration which is adapted to the personality of the user, and manufacturing the vehicle at least partly based on the determined vehicle configuration. The actual manufacturing step may be optional if the determined vehicle configuration is discarded and the vehicle is finally not manufactured, for example. While it is conceivable that the vehicle configuration is determined in this manner on the personality data of the user alone, it will be understood that further factors may be taken into consideration for the determination of the vehicle configuration. For example, the user may make at least one pre-selection regarding certain vehicle configuration options (e.g., selecting a certain model or a certain vehicle color) and the determination of the vehicle configuration may then be performed in dependence from the at least one pre-selection. Additionally or alternatively, recommendations from an online advisor (e.g., a human advisor or a virtual advisor, such as a chatbot) may be taken into consideration for the determination of the vehicle configuration. For example, the user may have an online discussion with an online advisor and the determination of the vehicle configuration may then be performed in dependence from one or more recommendations made by the online advisor. In the manufacturing process of the vehicle, it will be understood that the determined vehicle configuration may also affect the manufacturing of vehicle parts needed for the manufacturing of the vehicle. For example, manufacturing the vehicle may comprise manufacturing one or more vehicle parts to be used for manufacturing the vehicle, wherein the vehicle parts are manufactured (e.g., using a 3D printer) in accordance with the determined vehicle configuration.

In a generalization of the above use case, the technique presented herein may be used to determine a composition of a product that is adapted to the personality of the user prior to producing product, wherein the product may then be produced at least partly based on (or “in accordance with”) the determined composition. Such product may not only be a vehicle, as mentioned in the previous use case, but may also be a chemical or pharmaceutical product (e.g., a cosmetic product, such as a cream, including skin creams, etc.), a textile product or a food product, for example. The product may be producible in different composition options (e.g., as offered by a producing company). While a chemical or pharmaceutical product or food product may be producible with different ingredient options or ingredient composition options, a textile product may be producible with different textile material, clothing style or cut options, for example. When a user is about to order such product, the composition of the product may be determined to be specifically adapted to the personality of the user. For a cosmetic product, for example, at least one of a moisture level (e.g., moist/dry), a glossiness level (e.g., glossy/matte), a flavor type (e.g., with flavor/neutral), a fragrance type (e.g., with fragrance/neutral) and a skin effect type (e.g., skin-soothing/tingling) may be adapted to the personality of the user, for example. Based on the determined composition, the product may then be produced accordingly. As such, in line with the above description, it may also be envisaged a method for producing a product including an efficient retrieval of a digital representation of personality data of a user by a client device from a server, the digital representation of the personality data being processed at the client device to provide a composition of the product adapted to the personality of the user. The method may comprise sending, from the client device to the server, a request for a digital representation of personality data for a user, receiving, by the client device from the server, the requested digital representation of the personality data of the user, the personality data of the user being computed, based on input obtained from the user, using a neural network trained to compute personality data for a user based on input obtained from the user, processing the digital representation of the personality data to determine a composition of a product which is adapted to the personality of the user, and producing the product at least partly based on the determined composition. The producing step may be optional if the determined composition is discarded and the product is finally not produced, for example. While it is conceivable that the composition of the product is determined in this manner on the personality data of the user alone, it will be understood that further factors may be taken into consideration for the determination of the product. For example, the user may make at least one pre-selection regarding certain composition options (e.g., selecting a certain ingredient) and the determination of the composition may then be performed in dependence from the at least one pre-selection. Additionally or alternatively, recommendations from an online advisor (e.g., a human advisor or a virtual advisor, such as a chatbot) may be taken into consideration for the determination of the composition. For example, the user may have an online discussion with an online advisor and the determination of the vehicle configuration may then be performed in dependence from one or more recommendations made by the online advisor.

In a still further vehicle-related use case, the provision of the user-adapted service to the user may relate to security features that are directed to prevent damage from a user potentially having suicidal tendencies. To this end, the client device (e.g., the vehicle) may monitor predefined conditions (e.g., based on sensor measurements) which are potentially indicative of a suicidal intent of the user. If a suicidal intent is determined based on such conditions, the client device may compare the detected conditions with the personality data of the user and, if the combination of the detected conditions and the personality data of the user (e.g., indicating that the user suffers from strong depression) leads to the conclusion that a suicidal risk may indeed be given, preventive measures may be taken. The client device may thus be configured to provide a user-adapted service not only in consideration of the digital representation of the personality data of the user, but also in consideration of (or “based on”/“in accordance with”) predefined conditions being monitored and being potentially indicative of a suicidal intent of the user (the digital representation of the personality data of the user and the detected predefined conditions may in other words be combined prior to providing the user-adapted service to the user), wherein providing the user-adapted service to the user may comprise triggering one or more preventive measures counteracting a suicidal intent of the user. An exemplary condition may include detecting that the user keeps sitting or switches to a lying position in the vehicle while the vehicle's motor is still running, but the vehicle is not moving for at least a predetermined amount of time (potentially indicative of exhaust gas intrusion into the passenger cabin; this could optionally also be sensed by a sensor in the passenger cabin). Corresponding countermeasures may include at least one of triggering an alarm, triggering an emergency call (e.g., to a depression hotline, police, friends, family, etc.) or simply stopping the motor. Another predefined condition may include detecting that the user parks the vehicle at an area of suicidal risk, such as at a bridge, steep cliff, or aside a river or lake, which may likewise cause triggering an alarm or emergency call. A still further condition may include detecting the fact that the user tailgates in traffic while driving at high velocity, optionally combined with detection of screams in the passenger cabin indicative of an outburst of rage of the user, while detecting at the same time that the user is the sole passenger in the vehicle (e.g., using seat occupancy detection) to rule out that the screams may be a result of a dispute among several passengers. Corresponding countermeasures may include at least one of automatically reducing/limiting the vehicles' travel speed, automatically keeping a safety distance, starting an automated conversation or playing music to relax the user, and suggesting alternative travel routes, for example. It will be understood that these conditions and measures are merely exemplary and that various other use cases are generally conceivable.

In still another vehicle-related use case, the provision of the user-adapted service to the user may not only relate to the user's vehicle itself, but may relate to a whole swarm of vehicles. When a group of vehicles (including the user's vehicle) travels in vicinity to each other (e.g., in the range of vision) and when the personality data of the users (e.g., drivers/passengers) of the other vehicles is available as well (e.g., in the same/similar manner as described above for the present user itself), the personality data of the present user may be compared (or “matched”) with the personality data of the respective other drivers in order to determine and implement a collectively enhanced driving behavior of the group of vehicles, i.e., a driving behavior (or “configuration”) of the group of vehicles which enhances (or “optimizes”) traffic in consideration of (or “while respecting”) the individual driver's personalities, optionally in further consideration of additional driving goals or preferences or a mood of the respective drivers. The vehicle may thus be one of a plurality of vehicles traveling in vicinity to each other, wherein the digital representation of the personality data of the user may be compared with one or more digital representations of personality data of users of the other ones of the plurality of vehicles to implement a collectively enhanced driving behavior of the plurality of vehicles considering the individual personalities of the respective users, optionally further considering driving goals or preferences or a mood of the respective users. For example, if a group of vehicles travels using autopilot, it may be conceivable that a vehicle having a stressed driver may overtake another vehicle whose driver has a more relaxed personality that allows accepting such overtaking action. The collectively enhanced driving behavior may be directed to enhancing (or “optimizing”) the traffic flow or the energy consumption among the group of vehicles, for example. In a platoon of vehicles, it may thus be conceivable that vehicles with more relaxed drivers travel in the slipstream of other vehicles, or that electric vehicles traveling on a shorter distance journey and having sufficient electric energy transfer part of their energy (e.g., using induction) to other vehicles having more conservative drivers that travel on a longer distance journey. In order to consider particular driving goals or preferences or a mood of the users, the users may input corresponding goals or preferences or a mood, such as at the beginning or during the journey in the vehicle, e.g., by statements like “I am in a hurry”, “I am relaxed”, “I am under pressure”, etc. Such information may also be gathered based on answers to questions posed to the user reflecting the user's driving goals or preferences or mood. Exemplary questions are listed in Table 4 below. If more than one passenger is in a vehicle, the personality data of all passengers of the vehicle may be used to determine collective personality data representative of all passengers in the vehicle, which may then be compared with the personality data of the other vehicles. Determining the collective personality data may include averaging or weighting the vehicle's individual passenger's personality data and its values, for example. The same may apply to driving goals and preferences of the users, which may likewise be combined into collective goals and/or preferences for comparison with other vehicles. To implement the collectively enhanced driving behavior among the group of vehicles, the vehicles may communicate with each other using vehicle-to-vehicle (V2V) communication, for example, to coordinate themselves accordingly.

It will be understood that the above-mentioned concept of determining collective personality data may be generalized and employed independently from the above multiple vehicle use case. In fact, collective personality data may be defined for virtually any use case in which multiple users are using a user-adapted service together. Thus, if a plurality of users collectively use a user-adapted service, the personality data of all users may be combined to determine collective personality data representative of all users that collectively use the service. Determining the collective personality data may include averaging or weighting the individual user's personality data and its values, for example. Providing the user-adapted service may then be based on the collective personality data, i.e., processing the digital representation of the personality data may then include processing the digital representation of the collective personality data to provide a user-adapted service to the users.

It will further be understood that the above-mentioned concept of defining driving goals or preferences of users traveling in a plurality of vehicles may be generalized and employed independently from the above multiple vehicle use case. Such use case related goals and preferences may be defined for virtually any use case and, therefore, use case related goals and preferences may generally be employed when providing a user-adapted service to a user. The use case related goals and preferences of a user may specifically relate to the user-adapted service provided to the user. Such goals and preferences may herein also be denoted as “actual personality information” of the user as they specifically relate to the “actual” user-adapted service currently being (or about to be) provided to the user. As such, use case related goals and preferences are to be distinguished from the goals and preferences mentioned above in relation to the “input obtained from the user”. As described above, the input obtained from the user may correspond to answers to questions regarding at least one of personality, goals and motivations of the user (wherein questions relating to personality may correspond to questions on preferences of the user). While use case related goals and preferences may likewise be obtained from answers to questions posed to the user, such questions may correspond to questions which are specifically directed to the “actual” use case (i.e., the user-adapted service) and are dependent from the particular use case, whereas the questions mentioned above in relation to the “input obtained from the user” may correspond to general questions regarding “general” goals and preferences of the user, i.e., questions not specifically directed to the present use case or, in other words, independent from the present use case. It will be understood that goals and preferences may not be the only “actual personality information” but that other types of actual personality information are generally conceivable. One such example may be a current mood (e.g., also understood in the sense of a current “feeling” or “condition”) of the user at the time of providing the user-adapted service to the user, which may be considered for adapting the service specifically to the user as well. Information on the current mood of the user may likewise be obtained from answers to questions posed to the user.

Exemplary questions regarding use case related goals, preferences and/or moods are shown in the tables presented herein below, wherein Table 4 provides an exemplary listing of questions specifically relating to a vehicle ride use case, Table 5 provides an exemplary listing of questions specifically relating to a vehicle manufacturing use case, Table 6 provides an exemplary listing of questions specifically relating to a transport means seat allocation use case, and Table 7 provides an exemplary listing of questions specifically relating to an e-commerce use case (purchasable products). It will be understood that these sets of use case related questions are merely exemplary and that various other types of questions for these and other use cases are generally conceivable, as long as the questions are directed to use case related goals, preferences and/or moods in the above sense. From the exemplary sets of questions presented in Tables 4 to 7, it may easily be seen how these types of questions (specifically directed to the “actual” use case) distinguish from the questions on “general” goals and preferences of the user shown in Tables 2 and 3, which are use case independent.

In some implementations, the “actual personality information” may be used as the “input obtained from the user” in the methods described above in relation to FIGS. 2 and 3, either as sole “input obtained from the user” or in combination with any of the other above-described “input obtained from the user”. It may thus be envisaged a method for providing a user-adapted service to the user which may generally correspond to the methods described above in relation to FIGS. 2 and 3, the only difference being that the “actual personality information” may be used as (sole or additional) “input obtained from the user”, based on which the neural network may then compute the personality data of the user in line with the above description.

As said, the actual personality information of the user may be obtained from answers to questions posed to the user. In other variants, actual personality information, such as the current mood of the user and the use case specific preferences of the user, may not only be obtained from the user in a question/answer scheme. For example, the actual personality information may be obtained on the basis of body scan data in the sense of the above description. At least one of the current mood of the user and the one or more preferences of the user may thus be obtained from body scan data indicative of characteristics of the user derivable by scanning at least a portion of the body of the user. The body scan data may correspond to and may be obtained in line with the above description regarding the body scan data. In order to assess the current mood of the user, the emotional state of the user may be derived using one the techniques described above, for example, such as by interpreting a user's facial expression, gesture and/or voice using body/face/voice recognition techniques. Several items of body scan data may be combined conclude on the mood or on a preference of the user. In a vehicle, for example, sensors in a steering wheel may measure hand pressure, blood pressure and pulse in order to determine the stress level of a user with high accuracy. As another example, a hectic behavior of the user may be detected based on the time between unlocking the door, opening the door, taking seat behind the steering wheel, time to ignition, shift gears, etc. (each action detected by a different sensor in the vehicle). At least two different types of body scan data may thus be combined to determine the at least one of the current mood of the user and the one or more preferences of the user. Other variants of body scan data may be obtained based on eye-tracking, which may be used to detect a preference of the user, e.g., on the basis of an item looked at by the user more than a threshold amount of time. Eye-tracking data may likewise be combined with other body scan data, such as blood pressure/pulse measurements, for example, which may be indicative of whether the looked-at item causes an emotional change for the user. Other than eye-tracking, it will be understood that mouse-tracking could be used as an alternative technique, e.g., when the user uses a computer. At least one of the one or more preferences of the user may thus be obtained by eye-tracking or mouse-tracking the user.

In case a plurality of users collectively use the user-adapted service, it will be understood that the body scan data obtained for all individual users may be combined to determine collective body scan data representative of all users that collectively use the service, i.e., a user group. Determining the collective body scan data may include averaging or weighting the individual user's body scan data and its values, for example. The current collective mood of the user group and the collective preference of the user group may then be obtained from the collective body scan data. Providing the user-adapted service may then be based on the collective body scan data, i.e., processing the digital representation of the personality data may include processing the digital representation of the collective personality data to provide a user-adapted service to the user group, wherein the collective personality data is computed based on the collective body scan data. As a mere example, if it is detected by facial recognition that three out of four passengers in a vehicle are anxious in view of bad weather conditions while riding along a coastal serpentine route, the current collective mood of the whole group of passengers may be determined to be anxious and, as a consequence, the driving configuration of the vehicle may be adapted to be based on more safety features.

It will be understood that the technique presented herein may not only be employed in vehicle/transport means related use cases, but also in other use cases, such as to adapt the configuration of smart home appliances or robots to the personality of a user, for example. As such, in line with the above description, it may also be envisaged a method for adapting a configuration of a smart home appliance (e.g., automatic roller shutters, air conditions, refrigerators, washing machines, televisions, set-top boxes, etc.) including an efficient retrieval of a digital representation of personality data of a user, wherein the digital representation of the personality of the user may be processed at the client device to adapt a configuration of the smart home appliance to the personality of the user (e.g., to adapt the way in which the smart home appliance carries out its primary task, such as its shutting (roller shutters), heating/cooling (air conditions), refrigerating (refrigerators), washing (washing machines) or recording/display (televisions/set-top boxes) tasks). Similarly, in line with the above description, it may be envisaged a method for adapting a configuration of a robot (e.g., a humanoid robot, a domestic robot configured to carry out one or more household tasks, a robot acting as a virtual driver driving a vehicle, a vendor robot in a supermarket, an agricultural robot, a robotic exoskeleton, etc.) including an efficient retrieval of a digital representation of personality data of a user, wherein the digital representation of the personality of the user may be processed at the client device to adapt a configuration of the robot to the personality of the user (e.g., to adapt a behavior of the robot, such as the way in which the robot moves, performs a working procedure or performs control, like adapting the way how a humanoid robot mimics facial expressions (e.g., lip or eye movement), adapting the way in which household tasks are carried out by a domestic robot, adapting the way in which an agricultural robot performs planting tasks, or adapting the way in which a robotic exoskeleton supports the movements of a user carrying the exoskeleton).

Various other use cases are generally conceivable. Other use cases may comprise the adaptation of the configuration of virtual robots, the adaptation of the configuration of medical devices, or even the stimulation of a brain, for example. As such, in line with the above description, it may also be envisaged a method for adapting a configuration of a virtual robot (e.g., a chatbot, virtual service personnel or virtual personal assistant) including an efficient retrieval of a digital representation of personality data of a user, wherein the digital representation of the personality of the user may be processed at the client device to adapt a configuration of the virtual robot to the personality of the user (e.g., to adapt the way in which the virtual robot carries out its task of supporting the user). In some variants, a virtual robot may be presented in the form of a hologram (e.g., displayed in free space or as part of a head-up display, such as of a vehicle). While it is conceivable that the displayed hologram may reflect a person (e.g., an avatar) speaking with the user, it will be understood that other images or videos adapted to the personality of the user may be employed for the display of the hologram as well. Moreover, not only the display of a hologram may be adapted, but also the way in which the virtual robot interacts (e.g., speaks) with the user may be adapted, such as by adapting the voice characteristics of the virtual robot (e.g., voice frequency/volume, male/female, etc.) or the virtual robot's way of speaking. As a mere example, a hologram displayed in a head-up display of a vehicle could be displayed as a police officer that speaks with an authoritarian language. Adapting the configuration of a virtual robot may also relate to the way notices, instructions or warnings are provided to the user. Such messages may be provided to the user in a manner adapted to the personality of the user, so that e.g. the probability of behavioral deficits are reduced and/or the acceptance of the messages by the user is increased (e.g., by providing user-adapted statements explaining/justifying the provision of the message). In the vehicle context, it may be conceivable that a warning message directed to preventing the user from rubbernecking in case of a nearby accident is provided, if the user has a curious personality, for example, thereby potentially avoiding a further accident.

Similarly, in line with the above description, it may be envisaged a method for adapting a treatment plan for a patient or adapting a configuration of a medical device (e.g., a bedside medical device) including an efficient retrieval of a digital representation of personality data of a user, wherein the digital representation of the personality of the user may be processed at the client device to adapt a configuration of the medical device to the personality of the user, in particular to change the way a medical treatment is applied to the user, such as a treatment exerting physical forces on the body of the user and/or a treatment administering medical substances (e.g., drugs) to the user (e.g., to adapt the settings of a cardiac pacemaker, to adapt the mechanical configuration of an electromechanically adjustable prosthesis, to adapt a drug dispensing process or a dosage regime, such as the dosage of an analgesic, or the like). Similar to medical devices, it may be envisaged a method for adapting a configuration of a sports equipment (e.g., training devices, such as treadmills, fitness bikes, crosstrainers, etc.) including an efficient retrieval of a digital representation of personality data of a user, wherein the digital representation of the personality of the user may be processed at the client device to adapt a configuration of the sports equipment to the personality of the user (e.g., to adapt a resistance of the sports equipment to increase/decrease forces to be applied by the user, to adapt a position to be adopted by the user on the sports equipment, to adapt a training program stored on the sports equipment to the personality of the user, and the like). It will be understood that, as described above for virtual robots, adapting the configuration of a medical device or a sports equipment may likewise relate to the way notices, instructions or warnings are provided to the user (e.g., in order to make sure a drug is taken by the user at the appropriate time, or to motivate the user of a sports equipment during training in a manner most suitable to the user).

It will further be understood in this context that, more generally, any type of messages or information provided to the user as part of a user-adapted service (as generally described herein) may be adapted to the personality of the user, including advertising messages, for example. Such messages may be in some variants be displayed at remote screens in the vicinity (e.g., line of sight) of the user, such as at electronic advertisement panels (or “billboards”) installed at public locations (e.g., at an airport, on the street, etc.), for example. The client device (e.g., a smartphone or tablet carried by the user) may transmit the personality data to a server providing the user-adapted service (e.g., via a local network in which the client device is registered, such as a Wi-Fi network available at the public location), wherein the server may adapt the messages or information displayed at the remote screen to the user. It will be understood that, rather than through a Wi-Fi network, the personality data may also be transmitted to such server via other technical channels. In one variant, the personality data may be transmitted to the server together with a transaction carried out using the client device (e.g., a payment transaction for a purchase product or service), wherein the personality data may be conveyed to the server together with the transaction data, for example. In such variants, it may be conceivable to employ the personality data as a sort of “payment means” (or “currency”) for the transaction being completed. In other words, the user may be rewarded for granting access to the user's personality data by a certain (e.g., monetary) value, such as by offering a reduced (or even free of charge) rate (or “price”) of the product or service being purchased in exchange of the provision of the personality data by the user, for example.

It will be understood that providing messages or information to the user as part of a user-adapted service, as described above, may not only relate to advertising messages, but to any information provided to the user. For example, a user accessing an e-commerce service (e.g., visiting an e-commerce website or using an e-commerce app) may be presented with content (e.g., purchasable products) specifically adapted to the personality of the user. As another example, a user using an infotainment system in a transport means (e.g., vehicle, aircraft or train) may be presented with infotainment options (e.g., selectable movies, etc.) specifically adapted to the personality of the user. It will be understood that various other use cases of displaying user-adapted information to the user are generally conceivable. It is to be noted in this regard that not only the content may be displayed in a user-adapted manner, but also the look-and-feel of the displayed information. In one variant, displaying user-adapted information to the user may be implemented using a filter executed at the client device or the at least one other device which provides the service to the user (e.g., an end-user-device, such as a smartphone, tablet or laptop), wherein the filter may be executed locally at such device to filter out content based on the personality (e.g., preferences) of the user before the content is displayed to the user. As a mere example, if the content is provided to the user in the form of a website, the filter may be executed locally at the end-user device to remove content from the website, which the user is likely not interested in, before the content is displayed to the user at the device.

Another use case of providing information to the user as part of a user-adapted service may relate to communication applications. In communication applications, such as video telephony or chat applications, essential factors of common non-verbal communication that are normally recognizable when the communicating parties are physically present (e.g., factors like physical presence/energy, body posture, etc.) may be lost in digital communications. To alleviate such loss, it may be conceivable to adapt the display of a communication application (e.g., chat or video conferencing application) based on information on the personality data of a communicating partner, e.g., in a manner that enables the user to better understand the personality of the communicating partner and therefore enables the user to adapt his way of communicating to better comply with the personality of the communicating partner. In other words, personality data may be shared among communicating partners so that users may cope with their communicating partners in a more empathetic way and, therefore, the lost personal contact may be compensated (at least to a certain extent). Quality and effectivity of digital communication may thus be increased.

While it will be understood that the personality data being used to adapt the display of the communication application may correspond to the “raw value” of the user's personality data (in the sense described above), in a communication use case, it may be expedient that the personality data being used to adapt the display corresponds to a “comparative value” (or “relative value”) of the user's personality data (in the sense described above). More specifically, the personality data being used to adapt the display may correspond to the “comparative value” of the user's personality data in comparison to the personality data of the respective communicating partner. In one variant, adapting the display of the communication application based on the information on the personality data of the communicating partner may comprise displaying at least part of the personality data of the communicating partner (e.g., values of the user's personality dimensions or personality characteristics derived therefrom), enabling the user to better assess the counterpart's personality characteristics. In other variants, it may be conceivable to display words that should not be used in view of the personality of the communicating partner, or words that may positively be used and will likely cause a positive reaction of the communicating partner. In still other variants, adapting the display of the communication application may include adapting a video or background image shown to the user, wherein the video or background image may be specifically adapted to the personality of the user, e.g., to positively affect the user's attitude/feelings towards the communicating partner (as a mere example, in a video conference, the color of the counterpart's tie in the video image may be adapted to a color favored by the user). Not only the visible presentation, but also the audible presentation may be adapted to positively affect the user's attitude/feelings towards the communicating partner, e.g., by adapting the voice settings (e.g., voice frequency/volume, etc.) by which the counterpart is heard in accordance with the preferences of the user. It will be understood that such visual or audible adaptations may likewise be applied at the communicating partner's side.

It will be understood that, in certain contexts, the digital representation of the personality of the user may be stored—once it has been computed in accordance with one of the techniques presented herein, such as using the neural network based on input obtained from the user—on a chip card or on a device emulating a chip card (such as a smartphone emulating chip card functionality using NFC (Near Field Communication)), wherein the personality data of the user may be read from the chip card or device emulating a chip card before the personality data is processed at the client device to provide a user-adapted service to the user, as described above. It may thus be envisaged a method in which the above sending and receiving steps between the client device and the server (e.g., steps S302 and S304) may be omitted and, instead, the client device may read the digital representation of the personality data of the user from a chip card or device emulating a chip card, and then process the digital representation of the personality data to provide a user-adapted service to the user. Prior to storing the digital representation of the personality data on the chip card or device emulating a chip card, the digital representation of the personality data may have been computed, based on input obtained from the user, using a neural network trained to compute personality data for a user based on input obtained from the user, as generally described herein. In the medical context, for example, the digital representation of the personality of the user may be stored as part of a digital health record (or “digital patient file”) such that it may be automatically retrievable prior to the treatment of the patient, e.g., by reading the personality data from a chip card on which the digital health record is stored. The personality data retrieved from the chip card may then be processed to configure a medical device as described above, or to adapt any other medical service to the user, such allocating a hospital room to the user that suits the personality of the user, for example. Various other contexts in which such chip cards may be used are generally conceivable. As mere examples, the personality data may be stored on a bank card (e.g., and be processed to adapt a payment-related service to the user), an insurance card (e.g., and be processed to adapt an insurance product to the user), ana payback card (e.g., and be processed to adapt a payback offering to the user), or the like.

For purchasable products, it may also be conceivable to adapt the subsequent steps in the value chain to the personality of the user, such as the production of the product or its delivery. Providing the user-adapted service to the user may in these cases include adapting a production of a product and/or adapting a delivery of a product in accordance with the personality of the user. Thus, when the product is still to be produced after purchase, the production of the product may be specifically adapted to preferences of the user (e.g., a product printed using a 3D printer after purchase may be printed in a manner that is specifically adapted to the personality/preferences of the user). Likewise, in some variants, providing the user-adapted service to the user may include providing a logistics/delivery service specifically adapted to the personality of the user. For example, the packaging of a product (e.g., color or material of the packaging) may be specifically adapted to the personality/preferences of the user. Additionally or alternatively, the selected delivery technology (e.g., drone, delivery truck, bicycle courier) may be specifically adapted to the personality/preferences of the user (e.g., while elderly people may prefer receiving a delivery from a human being, younger people may prefer receiving a packet from a drone). Also, the delivery modalities (e.g., such as delivery time, delivery place and/or the priority of the delivery) may be specifically adapted to the personality of the user.

As explained above, the neural network as described herein may be seen as an efficient functional data structure which enables computing the requested personality data and providing the computed personality data in the form of a digital representation to a client device. With respect to feedback, it has been described that the neural network represents an efficiently updatable data structure which may be updated based on (arbitrary) feedback on the user's personality to refine its capability to compute the personality data. The neural network may as such be considered as a data structure which may be enriched by way of continuous learning on the basis of various feedback from a user so as to improve the reflection of the user's personality over time. In this way, and with the advent of ever-increasing computational resources in the next years and decades, the neural network may be considered to evolve as a copy of the user's mind capable of providing steadily increasing precision in the calculation of the user's personality the more feedback is fed into it. In a long-term perspective, it may be conceivable to build up a copy of a human mind that may allow querying the mind as if questions were posed to the user itself. A user's mind may thus be said to be “conserved” (at least to a certain extent). As explained above, the feedback by which the neural network is updated may be gathered at the client device and/or at the at least one device providing the service to the user. It will be understood, however, that still further devices may be employed to collect feedback on the personality of the user. In one such variant, it may be conceivable to use an implantable brain-computer-interface (e.g., as developed by Neuralink Corporation, http://neuralink.com/) to continuously collect feedback on the personality data of the user directly from the brain and update the neural network over time accordingly.

In some variants, a mind copy may then be used to adapt the behavior of a robot or virtual robot (e.g., the robots or virtual robots described above) by configuring them in accordance with the mind copy. In other words, a virtual representation of a brain may be fed into a robot or other form of intelligent system in order to influence the behavior of such system based on the personality of the user. As a mere example, a humanoid robot or a virtual robot (e.g., in the form of a virtual personal assistant or hologram) may be configured based on the mind copy to act as a copy of the actual human (as realistically as possible). The copy of the actual human may then be used to take over tasks that the actual human would normally do. As a mere example, it may be conceivable that the copy of the actual human conducts a telephone conversation as replacement for the actual human, without the counterpart of the conversation noticing that interaction does not take place with the actual human. Even further, it may be envisaged a method for stimulating a brain (e.g., of a living being or a virtual representation of a brain) including an efficient retrieval of a digital representation of personality data of a user, wherein the digital representation of the personality of the user may be processed at the client device to adapt a stimulation procedure for the brain based on the personality of the user. The stimulation procedure may comprise an electrical stimulation of a living being's brain or an adaptation/reconfiguration of a virtual representation of a brain, for example. In other use cases, as already indicated above, it may be conceivable to employ the mind copy, or more specifically the personality data in general, as a sort of “payment means” or “currency”, enabling the user to monetize his personality data, such as when carrying out a payment transaction, for example.

In all of the above-described examples and use cases, when it is referred to “adapting” a configuration or setting “to the personality of a user”, it will be understood that such adaptation may be implemented using predefined mappings that map a given characteristic of the user's personality (as indicated by the digital representation of the personality data of the user) to a particular configuration or setting of the corresponding device/apparatus (e.g., vehicle, transport means, smart home appliance, robot, medical device, etc., as described above). As said, for example, if the personality data indicates that a driver tends to be risk-averse, the driving mode of a vehicle may be set to economy or comfort, whereas for drivers that tend to have a risk-seeking personality, the driving mode may be set to sport mode. Such mappings may be predefined for each possible personality characteristic-configuration/setting combination and, depending on the obtained personality data of the user, the configuration or setting of the device/apparatus may be adapted accordingly. The mappings may be predefined at the client device, for example, and, if the client device configures at least one other device providing the service to the user, as described above, the client device may provide the predefined mappings to the at least one other device so that the mappings can be implemented on the at least one other device so as to provide the user-adapted service to the user. In this way, less computational burden may be put on the at least one other device, which may in other words act as the “mapping recipient” receiving the mappings from the client device, which may act as the “mapping provider”. It will be understood that, in other variants, the predefined mappings may also be predefined (or “prestored”) at the at least one other device, in which case the at least one other device may receive a given characteristic of the user's personality and map it to a particular configuration or setting of the at least one other device accordingly. The personality characteristic of the user may correspond to a value of a personality dimension (e.g., out of the Big Five) output by the neural network, as described above, for example.

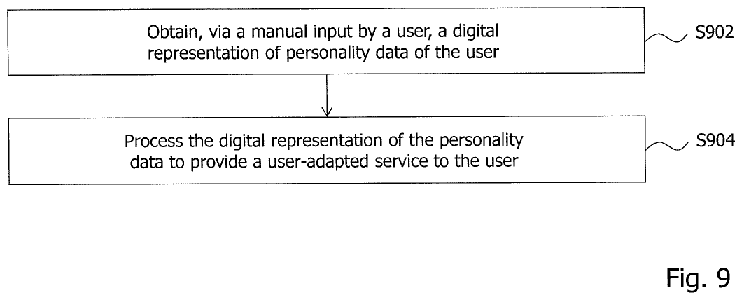

While, in the above description, the technique presented herein has been described as a technique for enabling efficient retrieval of a digital representation of personality data of a user by a client device from a server (which is employable in various use cases), it will be understood that the computed digital representation of the personality data of the user does not necessarily have to be sent to the client device directly from the server. Rather, the personality data of the user may, once available to the user, also be manually input to the client device by the user. On the side of the client device, it may thus also be envisaged a method for providing a user-adapted service to a user of a client device (this “client device” may not necessarily be understood in the sense of a device being in a client-server relationship because a direct client-server relationship may not exist in this case; the client device may thus also simply denoted as a “device”), wherein the method may be performed by the client device and may comprise obtaining, via a manual input by the user, a digital representation of the personality data of the user, and processing the digital representation of the personality data to provide a user-adapted service to the user. An illustration of such method is provided in FIG. 9, which shows, in step S902, a corresponding step of obtaining the digital representation of the personality data of the user and, in step S904, a corresponding step of processing the digital representation of the personality data of the user. Apart from the different way of inputting the digital representation of the personality data of the user to the client device (i.e., via a manual input instead of direct retrieval from the server), all aspects described above, especially with respect to the client device and the server, may apply to the method of FIG. 9 as well. The digital representation of the personality data of the user, which is obtained by the client device via the manual input of the user, may thus have been computed by a server, based on input obtained from the user, using a neural network trained to compute personality data for a user based on input obtained from the user (but does not necessarily have to be computed in this way, as it may also be conceivable that the manual input of the user corresponds to personality data of the user which has been determined in a different way). In one variant, in accordance with the above description, the client device may be a vehicle and providing the user-adapted service to the user may comprise adapting a driving configuration of the vehicle to a personality of the user.

As an alternative to manually inputting the digital representation of the personality data of the user, it may also be conceivable that the vehicle identification number is used to identify the selected vehicle configuration options (e.g., as offered by the vehicle manufacturer, as described above) based on which the vehicle has been manufactured. The thus identified vehicle configuration may then be used as the “input obtained from the user” in the above-described sense, i.e., to request the server to compute the personality data of the user using the neural network on the basis of the input. The thus obtained personality data of the user may then be used in any of the above-described ways to provide a user-adapted service to the user of the vehicle.

While the above embodiments have mostly been described with reference to an interaction between a client device and a server, e.g., including—from the viewpoint of the client device—the steps of sending (S302), to the server, a request for a digital representation of personality data for a user and receiving (S304), from the server, the requested digital representation of personality data of the user, it will be understood that the efficient retrieval of a digital representation of personality data proposed herein may not necessarily be implemented in such client/server scenario, but may more generally be expressed as a method for enabling efficient retrieval of a digital representation of personality data of a user, the digital representation of the personality data being processed to provide a user-adapted service to the user, the method comprising obtaining a digital representation of personality data of a user, the personality data of the user being computed, based on input obtained from the user, using a neural network trained to compute personality data for a user based on input obtained from the user, and processing the digital representation of the personality data to provide a user-adapted service to the user. Such more general formulation may be applied to all embodiments described above instead of the precise sending and receiving steps between a client device and a server.

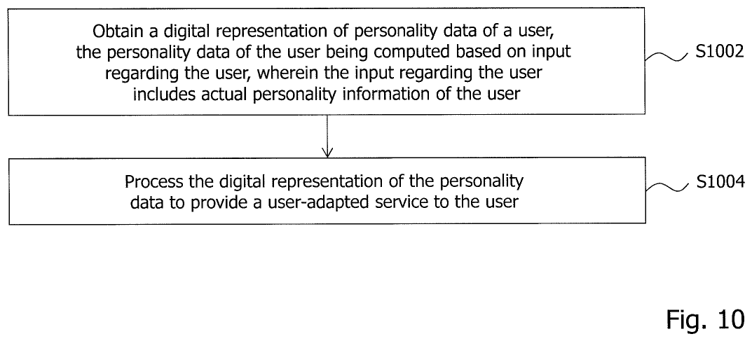

Referring to the above description regarding the “actual personality information”, it is noted that, in some implementations, the personality data of the user may be computed based on the actual personality information only, even without the use of a neural network. In such an implementation, which is exemplarily illustrated in FIG. 10, it may be envisaged a method for providing a user-adapted service to a user, the method being performed by a computing system and comprising obtaining, in step S1002, a digital representation of personality data of the user, the personality data of the user being computed based on input regarding the user, wherein the input regarding the user includes actual personality information of the user, the actual personality information of the user specifically relating to the user-adapted service and including at least one of a current mood of the user at the time of providing the user-adapted service to the user, one or more preferences of the user specifically relating to the user-adapted service, and one or more goals of the user specifically relating to the user-adapted service, and processing, in step S1004, the digital representation of the personality data to provide a user-adapted service to the user. It will be understood that the computing system may be formed by a client device and the server and, therefore, the obtaining step S1002 could likewise be realized in a client/server scenario in line with corresponding sending and receiving steps S302 and S304. As said, the actual personality information of the user may be obtained from answers to questions posed to the user. It will be understood that such computation of the personality data of the user may, in one variant, be performed using a proprietary algorithm (e.g., including mappings from the actual personality information the respective digital representations of the personality data of the user, etc.) but, in other variants, such computation may be performed in accordance with the above-described technique using a neural network. The personality data of the user may thus be computed based on the input regarding the user using a neural network trained to compute personality for a user based on input regarding the user. The input regarding the user may correspond to digital scores reflecting the answers to the questions posed to the user, wherein each digital score may be used as input to a separate input node of the neural network when computing the personality data of the user using the neural network. When the “actual personality information” is used as additional input in combination with the “input obtained from the user”, as described above, the input regarding the user may further correspond to digital scores reflecting answers to questions regarding at least one of personality, goals and motivations of the user, wherein likewise each digital score may be used as input to a separate input node of the neural network when computing the personality data of the user using the neural network, for example.

It is believed that the advantages of the technique presented herein will be fully understood from the foregoing description, and it will be apparent that various changes may be made in the form, constructions and arrangement of the exemplary aspects thereof without departing from the scope of the disclosure or without sacrificing all of its advantageous effects. Because the technique presented herein can be varied in many ways, it will be recognized that the disclosure should be limited only by the scope of the claims that follow.

TABLE 1
Questions regarding motivations of the user

| | |
|---|---|
| 1 | How important is this motive to you? - Peacefulness - a peaceful world without |
| | conflict |
| 2 | How important is this motive to you? - Equal treatment - everyone gets the same |
| | opportunities |
| 3 | How important is this motive to you? - Justice - everyone should be treated fairly |
| 4 | How important is this motive to you? - Duty - to work for a good cause (for example |
| | environment, other people) |
| 5 | How important is this motive to you? - Ethics - always doing what is right and good |
| 6 | How important is this motive to you? - Honesty - always being honest, never lying |
| 7 | How important is this motive to you? - Modesty - not demanding too much, satisfied |
| | with a little |
| 8 | How important is this motive to you? - Resoluteness - having and representing fixed |
| | values |
| 9 | How important is this motive to you? - Loyalty - be loyal to people / groups / |
| | organizations |
| 10 | How important is this motive to you? - Charity - helping those in need |
| 11 | How important is this motive to you? - Helpful - helping my fellow human beings |
| 12 | How important is this motive to you? - Selflessness - think of others first |
| 13 | How important is this motive to you? - Empathy - to put yourself in someone else's |
| | shoes |
| 14 | How important is this motive to you? - Listening - listening to others |
| 15 | How important is this motive to you? - To spread joy - to make others happy |
| 16 | How important is this motive to you? - Respected - to be respected by others |
| 17 | How important is this motive to you? - Trust - having the trust of others |
| 18 | How important is this motive to you? - Inspire - encourage and inspire others |
| 19 | How important is this motive to you? - Teach - teach others something |
| 20 | How important is this motive to you? - Be a role model - to be a role model for others |
| 21 | How important is this motive to you? - Salvation - find redemption |
| 22 | How important is this motive to you? - Faith - keep religious faith |
| 23 | How important is this motive to you? - Pleasing God - to live a godly life |
| 24 | How important is this motive to you? - Religious tradition - practicing religious / |
| | spiritual traditions |
| 25 | How important is this motive to you? - Soul growth - Spiritual / soul growth |
| 26 | How important is this motive to you? - Avoid impurity - avoid religiously impure |
| | actions |
| 27 | How important is this motive to you? - Harmony - achieving harmony with yourself |
| | and the universe |
| 28 | How important is this motive to you? - Meaning - find greater meaning / purpose in |
| | life |
| 29 | How important is this motive to you? - Wisdom - to be wise, to have a mature |
| | understanding of life |
| 30 | How important is this motive to you? - Growing personally - evolving / developing |
| | myself |
| 31 | How important is this motive to you? - Self-knowledge - to understand myself (better) |
| 32 | How important is this motive to you? - Self-loyal - stay true to yourself, not just |
| | follow the crowd |
| 33 | How important is this motive to you? - My feelings - to be in tune with my emotions |
| 34 | How important is this motive to you? - Acceptance - accepting myself and others as |
| | they are |
| 35 | How important is this motive to you? - Happiness - to be happy |
| 36 | How important is this motive to you? - Contentment - be satisfied with my life |
| 37 | How important is this motive to you? - Wellness - feel good within myself |
| 38 | How important is this motive to you? - Appreciating design - valuing beautiful form, |
| | shape, design |
| 39 | How important is this motive to you? - Appreciating art - know and appreciate art |
| 40 | How important is this motive to you? - Creativity - being creative (for example: |
| | artistically, scientifically, etc.) |
| 41 | How important is this motive to you? - Natural beauty - experience and enjoy the |
| | beauty of nature |
| 42 | How important is this motive to you? - Risk tolerance - willing and able to take risks |
| 43 | How important is this motive to you? - Curiosity - be and stay curious and interested |
| 44 | How important is this motive to you? - Uniqueness - being special or different |
| 45 | How important is this motive to you? - Thinking flexibly - taking different |
| | perspectives |
| 46 | How important is this motive to you? - Passion - pursuing things with passion |
| 47 | How important is this motive to you? - Strive for ideals - to realize desires / dreams / |
| | ideals |
| 48 | How important is this motive to you? - Playful - take life lightly and playfully |
| 49 | How important is this motive to you? - Spontaneity - be spontaneous |
| 50 | How important is this motive to you? - Adventurous - experience and explore new |
| | things |
| 51 | How important is this motive to you? - Exciting life - leading an exciting, stimulating |
| | life |
| 52 | How important is this motive to you? - Live today - fully savour the day |
| 53 | How important is this motive to you? - Leisure - have freetime and hobbies |
| 54 | How important is this motive to you? - Avoid fear - avoid frightening situations / do |
| | not face fears |
| 55 | How important is this motive to you? - Avoid stress - do not put yourself under |
| | pressure |
| 56 | How important is this motive to you? - Avoid feelings of guilt - do not feel guilty |
| 57 | How important is this motive to you? - Regret nothing - avoid feelings of remorse, do |
| | not look back |
| 58 | How important is this motive to you? - Avoid Injuries - do not risk bodily injury |
| 59 | How important is this motive to you? - Avoid criticism - try to avoid criticism by |
| | others |
| 60 | How important is this motive to you? - Avoid rejection - try to avoid rejection by |
| | others |
| 61 | How important is this motive to you? - Avoid conflicts - evade conflicts with others |
| 62 | How important is this motive to you? - Do no harm - do not hurt others' feelings, do |
| | not agitate anyone |
| 63 | How important is this motive to you? - Privacy - have alone time |
| 64 | How important is this motive to you? - Do not stand out - behave normally, so as not |
| | to attract attention |
| 65 | How important is this motive to you? - Avoid others - avoid contact with others |
| 66 | How important is this motive to you? - Do not strive - avoid work and effort |
| 67 | How important is this motive to you? - Avoid responsibility - be responsible for |
| | nothing |
| 68 | How important is this motive to you? - Procrastinate - push things back |
| 69 | How important is this motive to you? - Assertiveness - hold your ground, assert |
| | yourself |
| 70 | How important is this motive to you? - Sharing feelings - showing others my feelings |
| 71 | How important is this motive to you? - Communicate - look for conversation with |
| | others |
| 72 | How important is this motive to you? - Belonging - be part of a group and socially |
| | involved |
| 73 | How important is this motive to you? - Friends - a circle of friends as company |
| 74 | How important is this motive to you? - Close friends - have close friendships |
| 75 | How important is this motive to you? - New friends - making new friends |
| 76 | How important is this motive to you? - Reliability of others - have people you can |
| | rely on |
| 77 | How important is this motive to you? - Entertain others - make sure others have fun |
| 78 | How important is this motive to you? - Popular - be popular with others, be the center |
| | of attention |
| 79 | How important is this motive to you? - Sexy - be attractive, sexy / attract others |
| 80 | How important is this motive to you? - Sex - have sex / sexual experiences |
| 81 | How important is this motive to you? - Intimate connection - having an emotional and |
| | intimate relationship |
| 82 | How important is this motive to you? - Love - be in love, love someone |
| 83 | How important is this motive to you? - Hygiene - physical hygiene and care |
| 84 | How important is this motive to you? - Active - be active and busy |
| 85 | How important is this motive to you? - Control over environment - have a handle on |
| | things in my environment / apartment, etc. |
| 86 | How important is this motive to you? - Take time - take my time, do not rush |
| 87 | How important is this motive to you? - Fashion - place great emphasis on clothing and |
| | style |
| 88 | How important is this motive to you? - Attractive - look good, be pretty |
| 89 | How important is this motive to you? - Conventional - doing things in the traditional, |
| | conventional way |
| 90 | How important is this motive to you? - Security - feeling safe in life (money, |
| | profession, family) |
| 91 | How important is this motive to you? - Stability - regulated life and daily routine, no |
| | surprises |
| 92 | How important is this motive to you? - Be cared for - others / someone will take care |
| | of me |
| 93 | How important is this motive to you? - Have a mentor - have someone who shows me |
| | the direction in life |
| 94 | How important is this motive to you? - Competition - want to gauge myself with |
| | others and win |
| 95 | How important is this motive to you? - Be better - do things better than others |
| 96 | How important is this motive to you? - Control others - determine what others ought |
| | and ought not to do |
| 97 | How important is this motive to you? - Decide for others - make decisions for others |
| 98 | How important is this motive to you? - Revenge - seeking retribution |
| 99 | How important is this motive to you? - Receiving - employing others in order to give |
| | me what I want |
| 100 | How important is this motive to you? - Lead - specify the direction in groups |
| 101 | How important is this motive to you? - Influence - persuade / convince others |
| 102 | How important is this motive to you? - Enforce responsibility - hold people |
| | accountable |
| 103 | How important is this motive to you? - Exercise - physically active, on the move |
| 104 | How important is this motive to you? - Fitness - be physically fit and strong |
| 105 | How important is this motive to you? - Health - healthy diet, healthy body |
| 106 | How important is this motive to you? - Be physically able - to be able to do daily |
| | tasks |
| 107 | How important is this motive to you? - Athletic - be sporty / have athletic ability |
| 108 | How important is this motive to you? - Upbringing - be a good father / good mother |
| 109 | How important is this motive to you? - Bonding with children - having emotional |
| | attachment with my children |
| 110 | How important is this motive to you? - Family - stable family life |
| 111 | How important is this motive to you? - Good partnership - be happy in a relationship |
| 112 | How important is this motive to you? - Proximity to family - live close to parents, |
| | relatives |
| 113 | How important is this motive to you? - Help from family - receive family support |
| 114 | How important is this motive to you? - Obedience - do what my parents demand and |
| | require |
| 115 | How important is this motive to you? - Respect for elders - respect parents, |
| | grandparents |
| 116 | How important is this motive to you? - Master challenges - reach challenging goals |
| 117 | How important is this motive to you? - Overcoming setbacks - if I can not do |
| | something, I try again |
| 118 | How important is this motive to you? - Mastering things - doing something |
| | extraordinarily well |
| 119 | How important is this motive to you? - Ambitious - set ambitious goals and |
| | implement them with hard work |
| 120 | How important is this motive to you? - Competence - be competent in different areas |
| 121 | How important is this motive to you? - Do not fail - avoid failure |
| 122 | How important is this motive to you? - Perfection - strive for perfection |
| 123 | How important is this motive to you? - Confident - live with confidence and self- |
| | assurance |
| 124 | How important is this motive to you? - My judgment - trust in my own judgment |
| 125 | How important is this motive to you? - Control over life - have control over my life |
| 126 | How important is this motive to you? - Independent - independent and autonomous |
| | living |
| 127 | How important is this motive to you? - My principles - live according to my |
| | principles |
| 128 | How important is this motive to you? - Discipline - implementing projects |
| | purposefully and in a disciplined way |
| 129 | How important is this motive to you? - Master of my domain - determine / control my |
| | life |
| 130 | How important is this motive to you? - Reliability - I can be counted on |
| 131 | How important is this motive to you? - Logical - thinking and acting logically and |
| | rationally |
| 132 | How important is this motive to you? - Practical - pragmatic thinking, common sense |
| 133 | How important is this motive to you? - Thought out - think through and weigh |
| | decisions carefully |
| 134 | How important is this motive to you? - Attentive - be careful and attentive |
| 135 | How important is this motive to you? - Order - clean up, keep order |
| 136 | How important is this motive to you? - Manageable - I like it simple and |
| | straightforward |
| 137 | How important is this motive to you? - Plan - make plans, plan projects |
| 138 | How important is this motive to you? - Details - pay attention to details and trivialities |
| 139 | How important is this motive to you? - Correct - do things right, without mistakes |
| 140 | How important is this motive to you? - Efficient - on the direct path, without wasting |
| | time |
| 141 | How important is this motive to you? - On time - be punctual |
| 142 | How important is this motive to you? - Manage quickly - do things quickly |
| 143 | How important is this motive to you? - Analytical - analytical thinking |
| 144 | How important is this motive to you? - Technology - be technically savvy and work |
| | well with one's hands |
| 145 | How important is this motive to you? - Understand systems - understand how things |
| | and systems work |
| 146 | How important is this motive to you? - Intelligence - be smart |
| 147 | How important is this motive to you? - Intellectual - intellectual experiences and |
| | conversation |
| 148 | How important is this motive to you? - Education - have a good education |
| 149 | How important is this motive to you? - Educational achievement - achieve a higher |
| | education |
| 150 | How important is this motive to you? - Financial security - financially secure, without |
| | need |
| 151 | How important is this motive to you? - Financial need - can pay the bare minimum, |
| | make ends meet |
| 152 | How important is this motive to you? - Money - earn a lot of money |
| 153 | How important is this motive to you? - Estate - leave enough money for my children |
| 154 | How important is this motive to you? - Family provision - care for my family |
| 155 | How important is this motive to you? - Buy - I can buy what I want |
| 156 | How important is this motive to you? - Simple life - a simple, comfortable life |
| 157 | How important is this motive to you? - Success - be successful at work |
| 158 | How important is this motive to you? - Good job - have a good job |
| 159 | How important is this motive to you? - Occupation - have a job, not be unemployed |
| 160 | How important is this motive to you? - Professional development - be up to date in |
| | your career |
| 161 | How important is this motive to you? - Enjoying work - having a job I really like |

TABLE 2
Questions regarding goals of the user

| | |
|---|---|
| 1 | I can always manage to solve difficult problems if I try hard enough. |
| 2 | If someone opposes me, I can find the means and ways to get what I want. |
| 3 | It is easy for me to stick to my aims and accomplish my goals. |
| 4 | I am confident that I could deal efficiently with unexpected events. |
| 5 | Thanks to my resourcefulness, I know how to handle unforeseen situations. |
| 6 | I can solve most problems if I invest the necessary effort. |
| 7 | I can remain calm when facing difficulties because I can rely on my coping abilities. |
| 8 | When I am confronted with a problem, I can usually find several solutions. |
| 9 | If I am in trouble, I can usually think of a solution. |
| 10 | I can usually handle whatever comes my way. |
| 11 | Please name a goal that you have. |
| 12 | Please assign this goal to one of the following categories. |
| 13 | How achievable is this goal? |
| 14 | How confident are you that you will achieve your goal? |
| 15 | By when do you want to reach your goal? |
| 16 | How much time are you currently investing in achieving your goal? |
| 17 | How close are you to achieving your goal? |
| 18 | How easy / difficult is it for you to steadily work towards your goal? |
| 19 | Which measures do you use in order to achieve your goal? |
| 20 | Would something more positive be achieved by reaching your goal, or would |
| | something negative be avoided? |
| 21 | Now imagine that you have already reached your goal. Which of the following |
| | emotional states would this likely produce? |
| 22 | Now imagine, you would have failed in achieving your goal. Which of the following |
| | emotional states would this likely produce? |
| 23 | If I am being honest, I am primarily pursuing my goal... - ...because I want to prove to |
| | myself that I can accomplish things, master challenges and have competence in certain |
| | areas. |
| 24 | If I am being honest, I am primarily pursuing my goal... - ...because I want to act |
| | morally and support other people. |
| 25 | If I am being honest, I am primarily pursuing my goal... - ...because I want my life to |
| | have a higher purpose and meaning. |
| 26 | If I am being honest, I am primarily pursuing my goal... - ...because I want to be and |
| | stay healthy. |
| 27 | If I am being honest, I am primarily pursuing my goal... - ...because I want to fulfill |
| | myself through experience and exploration. |
| 28 | If I am being honest, I am primarily pursuing my goal... - ...because I want to have an |
| | intact family life. |
| 29 | If I am being honest, I am primarily pursuing my goal... - ...because I want to be |
| | dominant and lead others. |
| 30 | If I am being honest, I am primarily pursuing my goal... - ...because I want to be liked, |
| | wanted and socially recognized by others. |
| 31 | If I am being honest, I am primarily pursuing my goal... - ...because I want to be |
| | successful. |
| 32 | If I am being honest, I am primarily pursuing my goal... - ...because I want to grow |
| | religiously and spiritually. |
| 33 | If I am being honest, I am primarily pursuing my goal... - ...because I want to avoid |
| | conflicts and social rejection. |
| 34 | How important are the following resources to achieving your goal? - Skills and |
| | Abilities |
| 35 | How important are the following resources to achieving your goal? - Education and |
| | Knowledge |
| 36 | How important are the following resources to achieving your goal? - Social support in |
| | the private sphere (friends, family) |
| 37 | How important are the following resources to achieving your goal? - Practice / Training |
| 38 | How important are the following resources to achieving your goal? - Time |
| 39 | How important are the following resources to achieving your goal? - Financial |
| | resources |
| 40 | How important are the following resources to achieving your goal? - Social support |
| | through social media |
| 41 | How important are the following resources to achieving your goal? - Self-discipline / |
| | self-control / patience / endurance |
| 42 | How important are the following resources to achieving your goal? - Programs / |
| | products to support plan implementation (e.g., apps, fitness trackers, project planning |
| | tools) |
| 43 | How important are the following resources to achieving your goal? - Sufficient and |
| | detailed planning |
| 44 | To what degree can you improve in the following areas? - Relationship Management |
| | (communication skills, persuasion skills, negotiating skills, cooperation) |
| 45 | To what degree can you improve in the following areas? - Time Management |
| | (estimation of time required, strategy development, prioritizing, strategies for avoiding |
| | distractions) |
| 46 | To what degree can you improve in the following areas? - Self Management (self- |
| | monitoring, perseverance, strategies to get started) |
| 47 | To what degree can you improve in the following areas? - Resource Management |
| | (financial resources, materials, skills, competent individuals, knowledge, energy) |

TABLE 3
Questions regarding other personality aspects of the user

| | |
|---|---|
| 1 | Please specify your gender. |
| 2 | How old are you? |
| 3 | What is your nationality? |
| 4 | Please enter your current home postal code. |
| 5 | What is your current job? |
| 6 | Have you received a high school or equivalent diploma? |
| 7 | What is the highest degree you have received? |
| 8 | What is your marital status? |
| 9 | Do you smoke? |
| 10 | How often do you consume alcohol? |
| 11 | What is your favorite color? |
| 12 | What’s your favorite movie? |
| 13 | Which social media platform do you prefer? |
| 14 | How much time do you prefer spending on social media? |
| 15 | What is your dream job? |
| 16 | In a lake, there is a patch of lily pads. Everyday, the patch doubles in size. If it takes 48 |
| | days for the patch to cover the entire lake, how long would it take for the patch to cover |
| | half of the lake? |
| 17 | Jerry received both the 15th highest and the 15th lowest mark in the class. How many |
| | students are in the class? |
| 18 | A man buys a pig for $60, sells it for $70, buys it back for $80, and sells it finally for |
| | $90. How much has he made? |

TABLE 4
Questions regarding vehicle ride use case

| | |
|---|---|
| 1 | Mood - Are you relaxed or in a hurry? |
| 2 | Mood - Do you feel healthy or sick? |
| 3 | Mood - Are you happy or sad? |
| 4 | Preference - Do you like a comfortable ride experience or a |
| | sporty one? |
| 5 | Goal - How fast do you wish to get there? |
| 6 | Goal - How important is it for you to get there? |

TABLE 5
Questions regarding vehicle manufacturing use case

| | |
|---|---|
| 1 | Preference - What was your favorite car you owned? |
| 2 | Preference - What is your favorite car ever? |
| 3 | Preference - What are your payment preferences |
| | (leasing, financing, cash)? |
| 3 | Preference - Do you stop often or keep driving? |
| 4 | Preference - Do you switch lanes often? |
| 5 | Preference - Do you listen to music or radio during a ride? |
| 6 | Preference - Do you like new car smell? |
| 7 | Goal - How often will you use the car? |
| 8 | Goal - How long will you use the car? |
| 9 | Goal - Where will you use the car? |
| 10 | Goal - What distances will you be driving the car? |
| 11 | Goal - What image do you want to express (rich, tech, green)? |

TABLE 6
Questions regarding transport means seat allocation use case

| | |
|---|---|
| 1 | Mood - Are you relaxed or under pressure? |
| 2 | Mood - Do you feel healthy or sick? |
| 3 | Mood - Are you happy or sad? |
| 4 | Preference - Do you want to be left alone? |
| 5 | Preference - Do you want to get to know people? |
| 6 | Preference - Do you quickly feel stress or claustrophobic? |
| 7 | Preference - Do you quickly fall asleep? |
| 8 | Preference - Do you like to shop? |
| 9 | Preference - Do you want to eat or drink during the trip? |
| 10 | Preference - Do you want to watch entertainment during the trip? |
| 11 | Goal - How important is this trip for you? |
| 12 | Goal - Are you in a hurry to get your luggage? |

TABLE 7
Questions regarding e-commerce use case (purchase products)

| | |
|---|---|
| 1 | Mood - Did you have a good day? |
| 2 | Mood - Are you in love? |
| 3 | Mood - Do you feel stressed? |
| 4 | Preference - What is your favorite song? |
| 5 | Preference - What is your favorite movie? |
| 6 | Preference - What is your favorite book? |
| 7 | Preference - What is your favorite drive fast song? |
| 8 | Preference - What songs make you feel loved or happy? |
| 9 | Preference - What songs do you dislike? |
| 10 | Goal - Do you want to avoid plastic? |
| 11 | Goal - Do you support fair trade? |

Advantageous examples of the present disclosure can be phrased as follows:

1. A method for enabling efficient retrieval of a digital representation of personality data of a user (402) by a client device (502; 406) from a server (404), the digital representation of the personality data being processed at the client device (406) to provide a user-adapted service to the user (402), the method being performed by the server (404) and comprising:
storing (S202) a neural network (602) being trained to compute personality data of a user (402) based on input obtained from the user (402);
receiving (S204), from the client device (502; 406), a request for a digital representation of personality data for a user (402); and
sending (S206), to the client device (502; 406), the requested digital representation of the personality data of the user (402), wherein the personality data of the user (402) is computed using the neural network (602) based on input obtained from the user (402).
   2. The method of example 1, wherein the digital representation of the personality data of the user (402) is processed at the client device (502; 406) to configure at least one device (406) providing a service to the user (402), and, optionally:
wherein the at least one device (406) comprises the client device (406).
   3 The method of example 1 or 2, further comprising:
receiving feedback characterizing the user (402);
updating the neural network (602) based on the feedback; and
sending, to the client device (502; 406), a digital representation of updated personality data of the user (402), wherein the updated personality data of the user (402) is computed using the updated neural network (602), and, optionally:
wherein the digital representation of the updated personality data of the user (402) is processed at the client device (502; 406) to refine a configuration of the at least one device (406) providing the service to the user (402).
   4. The method of example 3, wherein the feedback includes behavioral data reflecting behavior of the user (402) monitored at the at least one device (406) when using the service provided by the at least one device (406), and, optionally:
wherein the behavioral data is monitored using measurements performed by the at least one device (406) providing the service to the user (402).
   5. The method of example 4, wherein the at least one device (406) comprises a vehicle and wherein the behavioral data comprises data reflecting a driving behavior of the user (402).
   6. The method of any one of examples 1 to 5, wherein the personality data of the user (402) is computed prior to receiving the request from the client device (502; 406) and wherein the request includes an access code previously provided by the server (404) to the user (402) upon computing the personality data of the user (402), the access code allowing the user (402) to access the digital representation of the personality data of the user (402) from different client devices (502; 406).
   7. The method of any one of examples 1 to 6, wherein the input obtained from the user corresponds to digital scores reflecting answers to questions regarding at least one of personality, goals and motivations of the user (402) and wherein each digital score is used as input to a separate input node of the neural network (602) when computing the personality data of the user (402) using the neural network (602).
   8. The method of example 7, wherein the questions correspond to questions selected from a set of questions representative of an optimally achievable result of computing personality data of a user (402), wherein the selected questions correspond to questions of the set of questions which are determined to be most influential with respect to the optimally achievable result, and, optionally:
wherein the number of the selected questions is less than 10% of the number of questions included in the set of questions.
   9. The method of example 8, wherein the questions are selected from the set of questions based on correlating results achievable by each single question of the set of questions with the optimally achievable result and selecting questions from the set of questions which have a highest correlation with the optimally achievable result, or
wherein the questions are selected iteratively from the set of questions, wherein, in each iteration, a next question is selected depending on an answer of the user to a previous question, wherein, in each iteration, the next question is selected as a question of the set of questions which is determined to be most influential on an achievable result for computing personality data of the user, and, optionally:
wherein the neural network (602) comprises a plurality of output nodes representative of a probability curve (604) of a result of the personality data of the user (402), wherein determining the most influential question of the set of questions as the next question of the respective iteration includes determining, for each input node of the neural network (602), a degree according to which a change in the digital score input to the respective input node of the neural network (602) changes the probability curve (604).
   10. A method for enabling efficient retrieval of a digital representation of personality data of a user (402) by a client device (502; 406) from a server (404), the method being performed by the client device (502; 406) and comprising:
sending (S302), to the server (404), a request for a digital representation of personality data for a user (402);
receiving (S304), from the server (404), the requested digital representation of the personality data of the user (402), the personality data of the user (402) being computed, based on input obtained from the user (402), using a neural network (602) trained to compute personality data for a user (402) based on input obtained from the user (402); and
processing (S306) the digital representation of the personality data to provide a user-adapted service to the user (402).
   11. A computer program product comprising program code portions for performing the method of any one of examples 1 to 10 when the computer program product is executed on one or more computing units.
   12. The computer program product of example 11, stored on one or more computer readable recording media.
   13. A server (100; 404) for enabling efficient retrieval of a digital representation of personality data of a user (402) by a client device (502; 406) from the server (404), the digital representation of the personality data being processed at the client device (502; 406) to provide a user-adapted service to the user (402), the server (404) comprising at least one processor (102) and at least one memory (104), the at least one memory (104) containing instructions executable by the at least one processor (102) such that the server (404) is operable to perform the method of any one of examples 1 to 9.
   14. A client device (110; 502; 406) for enabling efficient retrieval of a digital representation of personality data of a user (402) from a server (404), the client device (110; 502; 406) comprising at least one processor (112) and at least one memory (114), the at least one memory (114) containing instructions executable by the at least one processor (112) such that the client device (110; 502; 406) is operable to perform the method of example 10.
   15. A system comprising a server (100; 404) according to example 13 and at least one client device (110; 502; 406) according to example 14.
   16. A method for providing a user-adapted service to a user (402) of a vehicle (406), the method being performed by the vehicle (406) and comprising:
obtaining (S902), via a manual input of the user (402), a digital representation of personality data of the user (402); and
processing (S904) the digital representation of the personality data to provide a user-adapted service to the user (402),
wherein providing the user-adapted service to the user (402) comprises adapting a driving configuration of the vehicle (406) to a personality of the user (402).
   17. The method of example 16, wherein providing the user-adapted to the user (402) further comprises at least one of adapting an environmental condition in a passenger cabin of the vehicle (406) and adapting a user-specific setting regarding a passenger cabin of the vehicle (406) to the personality of the user (402).
   18. The method of example 16 or 17, wherein providing the user-adapted service to the user (402) is further performed in consideration of sensor data indicative of an attention level of the user (402) obtained in a passenger cabin of the vehicle (406).
   19. The method of any one of examples 16 to 18, wherein providing the user-adapted service to the user (402) is further performed in consideration of at least one of geographical data, weather data and time data regarding a planned route to be traveled using the vehicle.
   20. The method of any one of examples 16 to 19, wherein providing the user-adapted service to the user (402) is further performed in consideration of body scan data indicative of characteristics of the user (402) derivable by scanning at least a portion of the body of the user (402).
   21. The method of any one of examples 16 to 20, wherein providing the user-adapted service to the user (402) is further performed in consideration of predefined conditions being monitored and being potentially indicative of a suicidal intent of the user (402), wherein providing the user-adapted service to the user (402) further comprises triggering one or more preventive measures counteracting a suicidal intent of the user (402).
   22. The method of any one of examples 16 to 21, wherein the vehicle (406) is one of a plurality of vehicles (406) traveling in vicinity to each other, wherein the digital representation of the personality data of the user (402) is compared with one or more digital representations of personality data of users (402) of the other ones of the plurality of vehicles (406) to implement a collectively enhanced driving behavior of the plurality of vehicles (406) considering the individual personalities of the respective users (402), optionally further considering driving goals or preferences of the respective users (402).
   23. The method of any one of examples 16 to 22, wherein the personality data of the user (402) was computed by a server (404), based on input obtained from the user (402), using a neural network (602) trained to compute personality data for a user (402) based on input obtained from the user (402).
   24. The method of example 23, wherein the input obtained from the user (402) corresponds to digital scores reflecting answers to questions regarding at least one of personality, goals and motivations of the user (402) and wherein each digital score is used as input to a separate input node of the neural network (602) when computing the personality data of the user (402) using the neural network (602).
   25. The method of example 24, wherein the questions correspond to questions selected from a set of questions representative of an optimally achievable result of computing personality data of a user (402), wherein the selected questions correspond to questions of the set of questions which are determined to be most influential with respect to the optimally achievable result, and, optionally:
wherein the number of the selected questions is less than 10% of the number of questions included in the set of questions.
   26. The method of example 25, wherein the questions are selected from the set of questions based on correlating results achievable by each single question of the set of questions with the optimally achievable result and selecting questions from the set of questions which have a highest correlation with the optimally achievable result, or
wherein the questions are selected iteratively from the set of questions, wherein, in each iteration, a next question is selected depending on an answer of the user to a previous question, wherein, in each iteration, the next question is selected as a question of the set of questions which is determined to be most influential on an achievable result for computing personality data of the user, and, optionally:
wherein the neural network (602) comprises a plurality of output nodes representative of a probability curve (604) of a result of the personality data of the user (402), wherein determining the most influential question of the set of questions as the next question of the respective iteration includes determining, for each input node of the neural network (602), a degree according to which a change in the digital score input to the respective input node of the neural network (602) changes the probability curve (604).
   27. A computer program product comprising program code portions for performing the method of any one of examples 16 to 26 when the computer program product is executed on one or more computing units.
   28. The computer program product of example 27, stored on one or more computer readable recording media.
   29. A vehicle (406) for providing a user-adapted service to a user (402), the vehicle (406) comprising at least one processor (112) and at least one memory (114), the at least one memory (114) containing instructions executable by the at least one processor (112) such that the vehicle (406) is operable to perform the method of any one of examples 16 to 26.

1-14. (canceled)

15. A method for providing a user-adapted service to a user, the method comprising:
obtaining a digital representation of personality data of a user, the personality data of the user being computed based on input regarding the user, wherein the input regarding the user includes actual personality information of the user obtained from at least one answer to at least one question posed to the user, the at least one question specifically relating to a driving service of a vehicle to be provided to the user and including at least one of:
(a) one or more questions directed to a current mood of the user at a time of providing the driving service to the user,
(b) one or more questions directed to one or more preferences of the user specifically relating to the driving service to be provided to the user, and
(c) one or more questions directed to one or more goals of the user specifically relating to the driving service to be provided to the user; and
processing the digital representation of the personality data to provide a user-adapted service to the user, wherein providing the user-adapted service to the user comprises at least one of:
adapting a driving configuration of the vehicle to the user,
adapting an environmental condition in a passenger cabin of the vehicle to the user, and
adapting a user-specific setting regarding a passenger cabin of the vehicle to the user.

16. The method of claim 15, wherein at least one of the current mood of the user and the one or more preferences of the user are further obtained from body scan data indicative of characteristics of the user derivable by scanning at least a portion of the body of the user.

17. The method of claim 16, wherein at least two different types of body scan data obtained from the user are combined to determine the at least one of the current mood of the user and the one or more preferences of the user.

18. The method of claim 16, wherein at least one of the one or more preferences of the user is obtained by eye-tracking or mouse-tracking the user.

19. The method of claim 16, wherein, when a plurality of users collectively use the driving service, body scan data is obtained for all individual users of the plurality of users and combined to determine collective body scan data, wherein the driving service is provided based on the collective body scan data.

20. The method of claim 15, wherein the personality data of the user is computed based on the input regarding the user using a neural network trained to compute personality data for a user based on input regarding the user.

21. The method of claim 20, wherein the input regarding the user corresponds to digital scores reflecting the answers to the questions posed to the user and wherein each digital score is used as input to a separate input node of the neural network when computing the personality data of the user using the neural network.

22. The method of claim 21, wherein the input regarding the user further corresponds to digital scores reflecting answers to questions regarding at least one of personality, goals and motivations of the user and wherein each digital score is used as input to a separate input node of the neural network when computing the personality data of the user using the neural network.

23. The method of claim 21, wherein the questions correspond to questions selected from a set of questions representative of an optimally achievable result of computing personality data of a user, wherein the selected questions correspond to questions of the set of questions which are determined to be most influential with respect to the optimally achievable result, and, optionally:
wherein the number of the selected questions is less than 10% of the number of questions included in the set of questions.

24. The method of claim 23, wherein the questions are selected from the set of questions based on correlating results achievable by each single question of the set of questions with the optimally achievable result and selecting questions from the set of questions which have a highest correlation with the optimally achievable result, or
wherein the questions are selected iteratively from the set of questions, wherein, in each iteration, a next question is selected depending on an answer of the user to a previous question, wherein, in each iteration, the next question is selected as a question of the set of questions which is determined to be most influential on an achievable result for computing personality data of the user, and, optionally:
wherein the neural network comprises a plurality of output nodes representative of a probability curve of a result of the personality data of the user, wherein determining the most influential question of the set of questions as the next question of the respective iteration includes determining, for each input node of the neural network, a degree according to which a change in the digital score input to the respective input node of the neural network changes the probability curve.

25. One or more non-transitory computer readable recording mediums storing a computer program product executable by a computing device, the computer program product comprising:
obtaining instructions configured to cause obtaining of a digital representation of personality data of a user, the personality data of the user being computed based on input regarding the user, wherein the input regarding the user includes actual personality information of the user obtained from at least one answer to at least one question posed to the user, the at least one question specifically relating to a driving service of a vehicle to be provided to the user and including at least one of:
(a) one or more questions directed to a current mood of the user at a time of providing the driving service to the user,
(b) one or more questions directed to one or more preferences of the user specifically relating to the driving service to be provided to the user, and
(c) one or more questions directed to one or more goals of the user specifically relating to the driving service to be provided to the user; and
processing instructions configured to cause processing of the digital representation of the personality data to provide a user-adapted service to the user, wherein providing the user-adapted service to the user comprises at least one of:
adapting a driving configuration of the vehicle to the user,
adapting an environmental condition in a passenger cabin of the vehicle to the user, and
adapting a user-specific setting regarding a passenger cabin of the vehicle to the user.

26. A computing device comprising at least one processor and at least one memory, the at least one memory containing instructions executable by the at least one processor such that the computing device is operable at least to:
obtain a digital representation of personality data of a user, the personality data of the user being computed based on input regarding the user, wherein the input regarding the user includes actual personality information of the user obtained from at least one answer to at least one question posed to the user, the at least one question specifically relating to a driving service of a vehicle to be provided to the user and including at least one of:
(a) one or more questions directed to a current mood of the user at a time of providing the driving service to the user,
(b) one or more questions directed to one or more preferences of the user specifically relating to the driving service to be provided to the user, and
(c) one or more questions directed to one or more goals of the user specifically relating to the driving service to be provided to the user; and
process the digital representation of the personality data to provide a user-adapted service to the user, wherein providing the user-adapted service to the user comprises at least one of:
adapting a driving configuration of the vehicle to the user,
adapting an environmental condition in a passenger cabin of the vehicle to the user, and
adapting a user-specific setting regarding a passenger cabin of the vehicle to the user.
